(19) United States
(12) Patent Application Publication
(10) Pub. No.: US 2009/0299156 A1
(43) Pub. Date: Dec. 3, 2009

(54) CONTINUOUS MEDICAMENT SENSOR SYSTEM FOR IN VIVO USE

(75) Inventors: Peter C. Simpson, Encinitas, CA (US); Richard C. Yang, Irvine, CA (US); Bradley Shigeto Matsubara, Escondido, CA (US); Robert Boock, San Diego, CA (US); Eleanor McCarthy, Overland Park, KS (US); Mark Brister, Encinitas, CA (US)

Correspondence Address: KNOBBE, MARTENS, OLSEN & BEAR, LLP, 2040 MAIN STREET, FOURTEENTH FLOOR, IRVINE, CA, 92614, US

(73) Assignee: DexCom, Inc., San Diego, CA (US)

(21) Appl. No.: 12/365,683

(22) Filed: Feb. 4, 2009

Related U.S. Application Data
(60) Provisional application No. 61/030,179, filed on Feb. 20, 2008.

(51) Int. Cl. A61B 5/145; A61B 5/00

(52) U.S. Cl. 600/301; 600/309

(57) ABSTRACT

Systems and methods for continuous measurement of a medicament in vivo are provided. In some embodiments, the system is configured to provide information associated with medicament titration and includes a continuous analyte sensor and a communication device. In some embodiments, the system is configured for continuous ambulatory drug testing, including an ambulatory host monitor having a continuous sensor, a location module, a processor module and a transmitter. In some embodiments, the system is configured for continuously monitoring a hormone level and includes a continuous hormone sensor and a communication device configured to output hormone information in real time. Yet another embodiment provides an analyte sensor for continuous monitoring of a host's nutritional status, and is configured for both continuous glucose detection and continuous albumin detection.

### CROSS-REFERENCE TO RELATED APPLICATIONS

This application claims the benefit under 35 U.S.C. §119(e) of U.S. provisional application Ser. No. 61/030,179, filed Feb. 20, 2008, the disclosure of which is hereby expressly incorporated by reference in its entirety and is hereby expressly made a portion of this application.

### FIELD OF THE INVENTION

The preferred embodiments relate generally to continuous detection and/or measurement of analytes, such as but not limited to medicaments and hormones, in vivo.

### BACKGROUND OF THE INVENTION

Medicaments (e.g., drugs, alcohol) and bodily substances (e.g., hormones, metabolic products) are measured and/or monitored in a variety of settings. For example, in certain circumstances a medicament's therapeutic is undesirably narrow; which necessitates at least some titration of the medicament delivered to the host. For example, in a hospital setting, the amount of anticoagulant delivered to the host must be carefully regulated to prevent excessively long clotting times and host endangerment.

In some circumstances, drug testing is conducted in the context of employment, law-enforcement and/or rehabilitation of a drug addict. For example, drug of abuse testing is conducted on collected urine/blood samples, using a lateral-flow immunoassay device. It is well known that these testing procedures can be fraught with difficulty due to possible sample adulteration by the sample donor.

Similarly to drug testing, hormone testing is frequently conducted using lateral-flow immunoassay devices, especially in the home. Examples include the “pee-on-a-stick” pregnancy, ovulation and menopause testing devices available over the counter.

In some circumstances, a host can have impaired wound healing, which can be related to poor nutritional status. In such situations, glucose and albumin measurements can be performed regularly.

### SUMMARY OF THE INVENTION

In a first aspect, a system is provided for providing information associated with a titration of a medicament in a host, comprising: a continuous analyte sensor configured to detect a first signal associated with a medicament concentration in vivo in a host; and a communication device comprising an input module configured to receive titration parameters, and a processor module configured to process the first signal and the titration parameters to obtain titration information associated with a titration of the medicament, wherein the communication device is configured to output the titration information.

In an embodiment of the first aspect, the titration parameters comprise at least one parameter selected from the group consisting of medicament identity information, a target medicament concentration, a medicament concentration limit, a toxic medicament concentration, a medicament delivery rate, a medicament delivery time, host data, and medicament effect information.

In an embodiment of the first aspect, the processor module is configured to provide an alarm when the medicament concentration is substantially within a predetermined percentage of the medicament concentration limit.

In an embodiment of the first aspect, the titration information comprises at least one member selected from the group consisting of a current medicament concentration, a predicted medicament concentration, a change in medicament concentration, an acceleration of medicament concentration, a relationship of medicament concentration and a medicament concentration limit, rate of change information, a clearance rate, and a correlation between a medicament concentration and a medicament effect experienced by the host.

In an embodiment of the first aspect, the information comprises at least one member selected from the group consisting of a therapy recommendation and a therapy instruction.

In an embodiment of the first aspect, the input module is further configured to receive a second signal associated with an effect of the medicament, and wherein the processor module is further configured to process the first signal, the second signal and the titration parameters to obtain the titration information.

In an embodiment of the first aspect, the system further comprises a secondary medical device, for example, at least one device selected from the group consisting of a secondary analyte sensor and a patient monitor, wherein the secondary medical device is configured to detect a second signal associated with an effect of a delivered medicament.

In an embodiment of the first aspect, the effect of the delivered medicament is associated with a change in a host physical attribute.

In an embodiment of the first aspect, the medicament comprises an anti-multiple sclerosis medicament, and wherein the effect of the delivered medicament comprises a change in at least one member selected from the group consisting of a multiple sclerosis symptom and a side effect of the anti-multiple sclerosis medicament.

In an embodiment of the first aspect, the medicament comprises an anti-epilepsy medicament, and wherein the effect of the delivered medicament comprises a change in at least one member selected from the group consisting of an epilepsy symptom and a side effect of the anti-epilepsy medicament.

In an embodiment of the first aspect, the communication device is configured to output the titration information to a secondary medical device.

In an embodiment of the first aspect, the secondary medical device comprises an anesthesia device.

In an embodiment of the first aspect, the secondary medical device comprises a medicament delivery device.

In an embodiment of the first aspect, the secondary medical device is configured to monitor an attribute of the host.

In an embodiment of the first aspect, the processor module is configured to determine an optimal dose of the medicament.

In an embodiment of the first aspect, the communication device comprises a user interface configured to perform at least one of outputting the titration information and receiving titration parameters.

In a second aspect, a system is provided for continuous ambulatory drug testing, comprising: an ambulatory host monitor comprising a continuous sensor configured to detect a signal associated with a presence of a drug in vivo in a host, a location module configured to provide a location of the continuous sensor, and a first processor module configured to process the signal to obtain drug information; and a transmitter configured to transmit the drug information.

In an embodiment of the second aspect, the system further comprises a communication device located remotely from the ambulatory host monitor, wherein the communication device is configured to receive the drug information and the location, and to process the drug information and the location to obtain drug-monitoring information, and wherein the communication device is configured to output the drug-monitoring information.

In an embodiment of the second aspect, the drug-monitoring information comprises at least one of an instruction and a recommendation.

In an embodiment of the second aspect, the first processor module is configured to provide an alarm when the signal is below a programmed level.

In an embodiment of the second aspect, the drug is a drug of abuse and wherein drug information comprises information associated with a presence of the drug of abuse in the host.

In an embodiment of the second aspect, the drug is a medicament and the drug information comprises information associated with a presence of the medicament in the host.

In an embodiment of the second aspect, the medicament comprises an anti-tuberculosis medicament.

In an embodiment of the second aspect, the system further comprises a secondary device configured to operably connect with the ambulatory host monitor, wherein the ambulatory host monitor is further configured to provide drug information to the secondary device, and wherein the secondary device is configured to perform at least one of providing an alert and deactivating a machine.

In an embodiment of the second aspect, the continuous sensor is a transcutaneous continuous sensor.

In a third aspect, a system is provided for continuously monitoring a hormone level, comprising: a continuous hormone sensor configured to detect a signal associated with a hormone concentration in vivo in a host; and a communication device comprising a processor module configured to process the signal to provide hormone information, wherein the communication device is configured to output the hormone information in real time.

In an embodiment of the third aspect, the communication device is further configured to store the hormone information over time, and wherein the processor module is further configured to process the stored hormone information and the real-time hormone information to provide diagnostic information.

In an embodiment of the third aspect, the hormone is luteinizing hormone, and wherein the diagnostic information comprises a time period associated with ovulation in the host.

In an embodiment of the third aspect, the hormone is human chorionic gonadotropin, and wherein the diagnostic information comprises pregnancy information.

In an embodiment of the third aspect, the sensor is configured to measure a signal associated with at least one hormone selected from the group consisting of luteinizing hormone, estradiol, progesterone, follicle stimulating hormone, follicle stimulating hormone β subunit, thyroid stimulating hormone, testosterone, and human chorionic gonadotropin.

In a fourth aspect, an analyte sensor is provided for monitoring nutritional status in a host, comprising: a first sensing portion configured to measure a first signal associated with a glucose concentration in a host; a second sensing portion configured to measure a second signal associated with an albumin concentration in the host; and a processor module configured to process the first signal and the second signal to obtain nutrition information in vivo.

In an embodiment of the fourth aspect, the first sensing portion is configured and arranged to measure the first signal using at least one detection method selected from the group consisting of electrochemical detection, immunochemical detection, physical detection, optical detection, radiological detection, chemical detection, and combinations thereof.

In an embodiment of the fourth aspect, the second sensing portion is configured and arranged to measure the second signal using at least one detection method selected from the group consisting of electrochemical detection, immunochemical detection, physical detection, optical detection, radiological detection, chemical detection, and combinations thereof.

In an embodiment of the fourth aspect, the device further comprises an output module configured to output the nutrition information.

In an embodiment of the fourth aspect, the nutrition information comprises at least one member selected from the group consisting of an analyte concentration, a change in analyte concentration, a rate of change in analyte concentration, a peak analyte concentration, a lowest analyte concentration, a correlation between a glucose concentration and an albumin concentration, nutrition status, and an alarm.

### BRIEF DESCRIPTION OF THE DRAWINGS

FIG. 1 is a block diagram illustrating a continuous analyte sensor system 10, in one embodiment.

FIG. 2 is a block diagram illustrating an electronics configuration of a communication device 110, in one embodiment.

FIG. 3 is a flow chart 300 illustrating a method of medicament titration, in one embodiment.

FIG. 4 is a flow chart 400 illustrating a method of continuous ambulatory drug testing, in one embodiment.

FIG. 5 is a flow chart 500 illustrating a method of continuous hormone level monitoring, in one embodiment.

FIG. 6 is a flow chart 600 illustrating a method of continuous glucose and continuous albumin detection, in one embodiment.

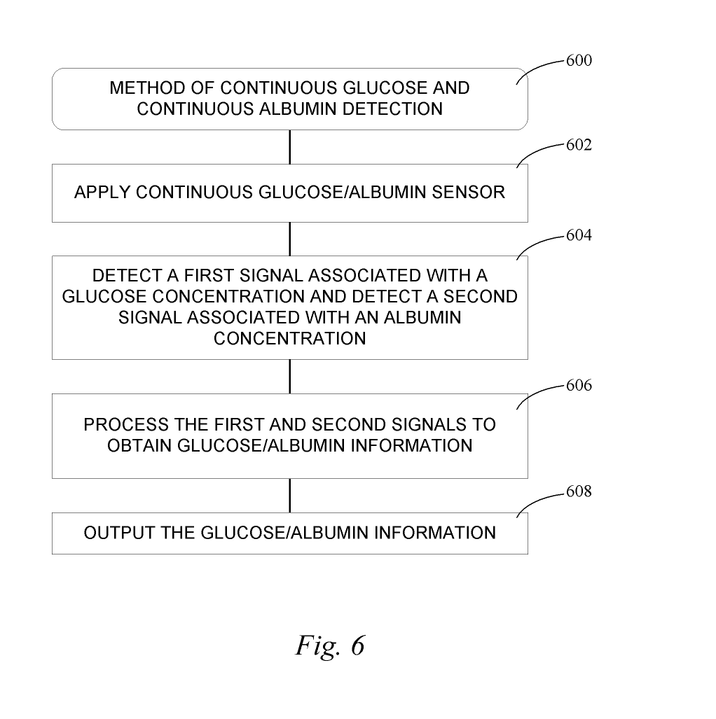

### DETAILED DESCRIPTION OF THE PREFERRED EMBODIMENT

The following description and examples illustrate a preferred embodiment of the present invention in detail. Those of skill in the art will recognize that there are numerous variations and modifications of this invention that are encompassed by its scope. Accordingly, the description of a preferred embodiment should not be deemed to limit the scope of the present invention.

### DEFINITIONS

In order to facilitate an understanding of the preferred embodiments, a number of terms are defined below.

The term “A/D Converter” as used herein is a broad term, and is to be given its ordinary and customary meaning to a person of ordinary skill in the art (and it is not to be limited to a special or customized meaning), and refers without limitation to hardware and/or software that converts analog electrical signals into corresponding digital signals.

The term “alarm,” as used herein is a broad term and is to be given its ordinary and customary meaning to a person of ordinary skill in the art (and is not to be limited to a special or customized meaning), and furthermore refers without limitation to a signal or indication related to an occurrence of an event and/or condition related to the host.

The term “analyte” as used herein is a broad term, and is to be given its ordinary and customary meaning to a person of ordinary skill in the art (and it is not to be limited to a special or customized meaning), and refers without limitation to a substance or chemical constituent in a biological fluid (for example, blood, interstitial fluid, cerebral spinal fluid, lymph fluid or urine) that can be analyzed. Analytes can include naturally occurring substances, artificial substances, metabolites, and/or reaction products. In some embodiments, the analyte for measurement by the sensor heads, devices, and methods disclosed herein is a medicament. However, other analytes are contemplated as well, including but not limited to acarboxyprothrombin; acylcarnitine; adenine phosphoribosyl transferase; adenosine deaminase; albumin; alpha-fetoprotein; amino acid profiles (arginine (Krebs cycle), histidine/urocanic acid, homocysteine, phenylalanine/tyrosine, tryptophan); andrenostenedione; antipyrine; arabinitol enantiomers; arginase; benzoylecgonine (cocaine); biotinidase; biopterin; c-reactive protein; carnitine; carnosinase; CD4; ceruloplasmin; chenodeoxycholic acid; chloroquine; cholesterol; cholinesterase; conjugated 1-β hydroxy-cholic acid; cortisol; creatine kinase; creatine kinase MM isoenzyme; cyclosporin A; d-penicillamine; de-ethylchloroquine; dehydroepiandrosterone sulfate; DNA (acetylator polymorphism, alcohol dehydrogenase, alpha 1-antitrypsin, cystic fibrosis, Duchenne/Becker muscular dystrophy, analyte-6-phosphate dehydrogenase, hemoglobinopathies A, S, C, and E, D-Punjab, beta-thalassemia, hepatitis B virus, HCMV, HIV-1, HTLV-1, Leber hereditary optic neuropathy, MCAD, RNA, PKU, Plasmodium vivax, sexual differentiation, 21-deoxycortisol); desbutylhalofantrine; dihydropteridine reductase; diptheria/tetanus antitoxin; erythrocyte arginase; erythrocyte protoporphyrin; esterase D; fatty acids/acylglycines; free β-human chorionic gonadotropin; free erythrocyte porphyrin; free thyroxine (FT4); free tri-iodothyronine (FT3); fumarylacetoacetase; galactose/gal-1-phosphate; galactose-1-phosphate uridyltransferase; gentamicin; analyte-6-phosphate dehydrogenase; glutathione; glutathione perioxidase; glycocholic acid; glycosylated hemoglobin; halofantrine; hemoglobin variants; hexosaminidase A; human erythrocyte carbonic anhydrase I; 17 alpha-hydroxyprogesterone; hypoxanthine phosphoribosyl transferase; immunoreactive trypsin; lactate; lead; lipoproteins ((a), B/A-1, β); lysozyme; mefloquine; netilmicin; phenobarbitone; phenyloin; phytanic/pristanic acid; progesterone; prolactin; prolidase; purine nucleoside phosphorylase; quinine; reverse tri-iodothyronine (rT3); selenium; serum pancreatic lipase; sissomicin; somatomedin C; specific antibodies (adenovirus, anti-nuclear antibody, anti-zeta antibody, arbovirus, Aujeszky's disease virus, dengue virus, Dracunculus medinensis, Echinococcus granulosus, Entamoeba histolytica, enterovirus, Giardia duodenalisa, Helicobacter pylori, hepatitis B virus, herpes virus, HIV-1, IgE (atopic disease), influenza virus, Leishmania donovani, leptospira, measles/mumps/rubella, Mycobacterium leprae, Mycoplasma pneumoniae, Myoglobin, Onchocerca volvulus, parainfluenza virus, Plasmodium falciparum, poliovirus, Pseudomonas aeruginosa, respiratory syncytial virus, rickettsia (scrub typhus), Schistosoma mansoni, Toxoplasma gondii, Trepenoma pallidium, Trypanosoma cruzi/rangeli, vesicular stomatis virus, Wuchereria bancrofti, yellow fever virus); specific antigens (hepatitis B virus, HIV-1); succinylacetone; sulfadoxine; theophylline; thyrotropin (TSH); thyroxine (T4); thyroxine-binding globulin; trace elements; transferrin; UDP-galactose-4-epimerase; urea; uroporphyrinogen I synthase; vitamin A; white blood cells; and zinc protoporphyrin. Salts, sugar, protein, fat, vitamins, and hormones naturally occurring in blood or interstitial fluids can also constitute analytes in certain embodiments. The analyte can be naturally present in the biological fluid, for example, a metabolic product, a hormone, an antigen, an antibody, and the like. Alternatively, the analyte can be introduced into the body, for example, a contrast agent for imaging, a radioisotope, a chemical agent, a fluorocarbon-based synthetic blood, or a drug or pharmaceutical composition, including but not limited to insulin; ethanol; cannabis (marijuana, tetrahydrocannabinol, hashish); inhalants (nitrous oxide, amyl nitrite, butyl nitrite, chlorohydrocarbons, hydrocarbons); cocaine (crack cocaine); stimulants (amphetamines, methamphetamines, Ritalin, Cylert, Preludin, Didrex, PreState, Voranil, Sandrex, Plegine); depressants (barbituates, methaqualone, tranquilizers such as Valium, Librium, Miltown, Serax, Equanil, Tranxene); hallucinogens (phencyclidine, lysergic acid, mescaline, peyote, psilocybin); narcotics (heroin, codeine, morphine, opium, meperidine, Percocet, Percodan, Tussionex, Fentanyl, Darvon, Talwin, Lomotil); designer drugs (analogs of fentanyl, meperidine, amphetamines, methamphetamines, and phencyclidine, for example, Ecstasy); anabolic steroids; and nicotine. The metabolic products of drugs and pharmaceutical compositions are also contemplated analytes. Analytes such as neurochemicals and other chemicals generated within the body can also be analyzed, such as, for example, ascorbic acid, uric acid, dopamine, noradrenaline, 3-methoxytyramine (3MT), 3,4-dihydroxyphenylacetic acid (DOPAC), homovanillic acid (HVA), 5-hydroxytryptamine (5HT), 5-hydroxyindoleacetic acid (FHIAA), and glucose.

The phrase “anti-epilepsy medicament” as used herein is a broad term, and is to be given its ordinary and customary meaning to a person of ordinary skill in the art (and it is not to be limited to a special or customized meaning), and refers without limitation to a medicament that can be used to alleviate the symptoms of, to treat and/or to cure epilepsy. Epilepsy drugs include, but are not limited to, acetazolamide, carbamazepine, clonazepam, clorazepate dipotassium, diazepam, divalproex sodium, ethosuximide, felbamate, fosphenyloin sodium, gabapentin, lamotrigine, levetiracetam, lorazepam, oxcarbazepine, phenobarbital, phenyloin, phenyloin sodium, pregabalin, primidone, tiagabine hydrochloride, topiramate, trimethadione, valproic acid, zonisamide, and their respective metabolites.

The phrase “anti-multiple sclerosis medicament” as used herein is a broad term, and is to be given its ordinary and customary meaning to a person of ordinary skill in the art (and it is not to be limited to a special or customized meaning), and refers without limitation to a medicament that can be used to alleviate the symptoms of, to treat and/or to cure multiple sclerosis. Anti-multiple sclerosis medicaments include, but are not limited to Corticosteroids (oral prednisone and intravenous methylprednisolone), Interferons (Betaseron, Avonex and Rebif) Glatiramer (Copaxone), Natalizumab (Tysabri), Mitoxantrone (Novantrone), and metabolites thereof.

The term “attribute” as used herein is a broad term, and is to be given its ordinary and customary meaning to a person of ordinary skill in the art (and it is not to be limited to a special or customized meaning), and refers without limitation to a quality, property, or characteristic of somebody or something. The term “physical attribute” can be used to refer to any characteristic of a host's body, such as but not limited to sex, weight, age, height, vital signs (e.g., temperature, blood pressure, heart rate, respiration rate), end tidal CO₂, glucose level, skin color, lung function, intracranial pressure, mental state, pain, neurological response to stimulation, a physical manifestation of a disease or illness experienced by the host, an effect of a drug experienced by the host, and the like.

The term “concentration” as used herein is a broad term, and is to be given its ordinary and customary meaning to a person of ordinary skill in the art (and are not to be limited to a special or customized meaning), and refers without limitation to a quantity of a substance per volume or weight. For example, in some circumstances, the amount of a substance in a bodily fluid (e.g., blood, plasma, serum, lymph, intracellular fluid, cerebrospinal fluid, etc.) is denoted as a weight/mass of the substance per unit of volume (e.g., mg/dl, mcg/ml). In another example, some medicaments can be provided for use (e.g., by the manufacturer, by a pharmacy, etc.) as a solution/suspension having a defined initial concentration (e.g., concentration as provided by the manufacturer, concentration in the container provided, the concentration prior to dilution).

The terms “continuous” and “continuously” as used herein are broad terms, and are to be given their ordinary and customary meanings to a person of ordinary skill in the art (and are not to be limited to a special or customized meaning), and refer without limitation to the condition of being marked by substantially uninterrupted extension in space, time or sequence. In one embodiment, an analyte concentration is measured continuously, continually, and/or intermittently (regularly or irregularly) for example at time intervals ranging from fractions of a second up to, for example, 1, 2, 5, or 10 minutes, or longer. For example, continuous cardiac marker measurement systems generally continually measure cardiac marker concentration without required user initiation and/or interaction for each measurement. These terms include situations wherein data gaps can exist (e.g., when a continuous sensor is temporarily not providing data, or when data from the continuous sensor is disregarded or not considered).

The phrase “continuous analyte sensing” as used herein is a broad term, and is to be given its ordinary and customary meaning to a person of ordinary skill in the art (and it is not to be limited to a special or customized meaning), and refers without limitation to the period in which monitoring of analyte concentration is continuously or continually performed, for example, at time intervals ranging from fractions of a second up to, for example, 1, 2, 5, or 10 minutes, or longer.

The term “counts” as used herein is a broad term, and is to be given its ordinary and customary meaning to a person of ordinary skill in the art (and it is not to be limited to a special or customized meaning), and refers without limitation to a unit of measurement of a digital signal. In one example, a raw data stream measured in counts is directly related to a voltage (for example, converted by an A/D converter), which is directly related to current from a working electrode.

The term “communication device,” as used herein is a broad term and is to be given its ordinary and customary meaning to a person of ordinary skill in the art (and is not to be limited to a special or customized meaning), and furthermore refers without limitation to a device configured to communicate information. In some embodiments, the output is to a display (bedside or remote therefrom).

The terms “computer” or “computer system” as used herein are broad terms, and are to be given their ordinary and customary meanings to a person of ordinary skill in the art (and are not to be limited to a special or customized meaning), and refer without limitation to a machine that can be programmed to manipulate data.

The term “criterion” and “criteria,” as used herein are broad terms and are to be given their ordinary and customary meaning to a person of ordinary skill in the art (and are not to be limited to a special or customized meaning), and furthermore refer without limitation to a basis for comparison; a reference point or set of points against which other things can be evaluated. In some embodiments, a criterion is associated with an action, instruction, command, and the like, that the system performs and/or provides when a criterion has been (or has not been) met. As a non-limiting example, the system can be configured such that when the concentration of a medicament meets a programmed criterion (e.g., the measured concentration is within 25%, 15%, 10%, or 5% of a maximum or minimum concentration) an alarm is sounded. In other embodiments, the criterion has two or more conditions that must be met before the associated action is taken. In some embodiments, the system is configured to compare data to two or more criteria, wherein each criterion is associated with a task to be performed. In some embodiments, a plurality of “criteria” must be met, wherein each of the criteria includes one or more conditions. For example, if conditions A and B have been satisfied, then alarm #1 is sounded, while, if condition C is met, then a text message is sent to a remote monitoring station. In some embodiments, a criterion has a single condition that must be met.

The phrase “drug of abuse” (DOA), as used herein is a broad term, and is to be given its ordinary and customary meaning to a person of ordinary skill in the art (and are not to be limited to a special or customized meaning), and refers without limitation to a substance, such as a drug (including its metabolites), alcohol, nicotine, or the toxins of certain plants/fungi, that is taken inappropriately or which may be habit forming. In some circumstances, a DOA is a drug, alcohol, toxin, and the like, taken for non-medicinal reasons, such as for psychoactive and/or performance enhancing effects, such as for a non-therapeutic or non-medical effect. In some circumstances, a DOA is a substance taken for a medical effect, wherein the consumption has become excessive or inappropriate (e.g., pain medications, sleep aids, anti-anxiety medication, Ritalin, erectile-dysfunction medications, and the like). A DOA can be an illicit (e.g., illegal) drug, an over-the-counter medication, a prescription medicament, and/or a legally consumable substance such as alcohol. In some circumstances, drug abuse can lead to physical and/or mental damage and (with some substances) dependence and addiction. DOAs can be discussed in the context of “substance abuse,” which refers without limitation to the overindulgence in and/or dependence of a drug or other chemical, leading to effects that may be detrimental to the individual's physical and mental health, or the welfare of others. In some circumstances, substance abuse includes consumption of a prescription medication by a person other than the person for whom the medication was prescribed.

The term “effect” as used herein is a broad term, and is to be given its ordinary and customary meaning to a person of ordinary skill in the art (and are not to be limited to a special or customized meaning), and refers without limitation to the result or consequence of an action. The effect(s) of medicament consumption may be desirable or undesirable, depending upon the circumstances. For example, the desirable effects of aspirin (acetylsalicylic acid) consumption can include pain relief, fever reduction, inflammation reduction and/or blood thinning. However, aspirin consumption can have undesirable effects, such as tinnitus (ringing in the ears), gastrointestinal distress and/or bleeding, increased clotting times, anaphylaxis and/or an increased risk of Reye's syndrome.

The term “electronics” as used herein is a broad term, and is to be given its ordinary and customary meaning to a person of ordinary skill in the art (and is not to be limited to a special or customized meaning), and refers without limitation to electronic circuitry configured to measure, process, receive, and/or transmit data.

The term “fluid delivery device,” as used herein is a broad term and is to be given its ordinary and customary meaning to a person of ordinary skill in the art (and is not to be limited to a special or customized meaning), and furthermore refers without limitation to a device configured to deliver a fluid to the host, such as a pump (e.g., a pump system) configured to deliver fluid and/or medicament(s) to a host via a catheter.

The term “hormone” as used herein is a broad term, and is to be given its ordinary and customary meaning to a person of ordinary skill in the art (and are not to be limited to a special or customized meaning), and refers without limitation to a chemical messenger that carries a signal from one cell (or group of cells) to another. In general, hormones regulate the function of their target cells (i.e., cells that express a receptor for the hormone). The action or net effect of a hormone is determined by a number of factors including the hormone's pattern of secretion and the response of the receiving tissue. Endocrine hormone molecules are secreted (released) directly into the bloodstream, while exocrine hormones (or ectohormones) are secreted directly into a duct, and from the duct they either flow into the bloodstream or they flow from cell to cell by diffusion in a process known as paracrine signaling. Vertebrate hormones fall into three chemical classes: amine-derived hormones (derivatives of tyrosine and tryptophan), peptide hormones (long and/or short amino acid chains, including proteins), and lipid and phospholipid-derived hormones.

The term “host” as used herein is a broad term, and is to be given its ordinary and customary meaning to a person of ordinary skill in the art (and it is not to be limited to a special or customized meaning), and refers without limitation to plants or animals, for example humans.

The term “medical device” as used herein is a broad term, and is to be given its ordinary and customary meaning to a person of ordinary skill in the art (and are not to be limited to a special or customized meaning), and refers without limitation to an instrument, apparatus, implement, machine, contrivance, implant, in vitro reagent, or other similar or related article, including a component part or accessory which is intended for use in the diagnosis of disease or other conditions, or in the cure, mitigation, treatment, or prevention of disease, in man or other animals, or intended to affect the structure or any function of the body of man or other animals.

The term “medicament” as used herein is a broad term, and is to be given its ordinary and customary meaning to a person of ordinary skill in the art (and are not to be limited to a special or customized meaning), and refers without limitation to a substance or agent (e.g., medicine, drug, medicinal application, or remedy) that treats, prevents and/or alleviates the symptoms of disease and/or illness. Depending upon its formulation, a medicament can be delivered to a host by any means, such as but not limited to injection, infusion, oral consumption, inhalation and/or topical application. In some circumstances, certain medicaments may be abused by a host, and thus may be referred to as drugs of abuse (DOAs). For example, some prescription sleep aids and analgesics can be addictive, and are sometimes abused by a patient prescribed such as medication.

The terms “operably connected” and “operably linked” as used herein are broad terms, and are to be given their ordinary and customary meaning to a person of ordinary skill in the art (and they are not to be limited to a special or customized meaning), and refer without limitation to one or more components being linked to another component(s) in a manner that allows transmission of signals between the components. These terms are broad enough to include wired and wireless connectivity.

The term “output,” as used herein is a broad term and is to be given its ordinary and customary meaning to a person of ordinary skill in the art (and is not to be limited to a special or customized meaning), and furthermore refers without limitation to presentation of data by the present system, such as (but not limited to) to the host, a caretaker, a component of the system or a secondary device integrated with the system. Output can include, but is not limited to, raw data, processed data, medicament information, titration information, drug monitoring information, hormone information, nutrition information, instructions and/or recommendations to the host, a caretaker (sometimes referred to as a “user” herein) or a secondary device, alerts, alarms, and the like. In some circumstances, data and/or information received from (or input by) the host, a caretaker, and/or a secondary device can be output by the system.

The term “potentiostat” as used herein is a broad term, and is to be given its ordinary and customary meaning to a person of ordinary skill in the art (and it is not to be limited to a special or customized meaning), and refers without limitation to an electrical system that applies a potential between the working and reference electrodes of a two- or three-electrode cell at a preset value and measures the current flow through the working electrode. A potentiostat can include multiple channels, such that potentials can be applied to two or more working electrode-reference electrode pairs. Typically, the potentiostat forces whatever current is necessary to flow between the working and reference or counter electrodes to keep the desired potential, as long as the needed cell voltage and current do not exceed the compliance limits of the potentiostat.

The terms “processor module” and “processor” as used herein are broad terms, and are to be given their ordinary and customary meaning to a person of ordinary skill in the art (and are not to be limited to a special or customized meaning), and refer without limitation to a computer system, state machine, processor, and the like designed to perform arithmetic or logic operations using logic circuitry that responds to and processes the basic instructions that drive a computer.

The terms “raw data stream” and “data stream” signal as used herein are broad terms, and are to be given their ordinary and customary meaning to a person of ordinary skill in the art (and they are not to be limited to a special or customized meaning), and refer without limitation to an analog or digital signal directly related to the analyte concentration measured by the analyte sensor. In one example, the raw data stream is digital data in “counts” converted by an A/D converter from an analog signal (for example, voltage or amps) representative of an analyte concentration. The terms broadly encompass a plurality of time spaced data points from a substantially continuous analyte sensor, which comprises individual measurements taken at time intervals ranging from fractions of a second up to, for example, 1, 2, or 5 minutes or longer. In some embodiments, raw data includes one or more values (e.g., digital value) representative of the current flow integrated over time (e.g., integrated value), for example, using a charge counting device, or the like.

The term “RF transceiver” as used herein is a broad term, and is to be given its ordinary and customary meaning to a person of ordinary skill in the art (and it is not to be limited to a special or customized meaning), and refers without limitation to a radio frequency transmitter and/or receiver for transmitting and/or receiving signals.

The term “secondary device,” as used herein is a broad term and is to be given its ordinary and customary meaning to a person of ordinary skill in the art (and is not to be limited to a special or customized meaning), and furthermore refers without limitation to a device distinct from a primary device. In some circumstances, the secondary device can be a medical device (also referred to as a secondary medical device), such as but not limited to any type of patient monitor, fluid delivery device (e.g., for delivery of IV medicaments, fluids and nutrition), or a medical device to assist the host in a bodily function (e.g., a ventilator assists the host in breathing when the host is not able to adequately perform that function alone). In some circumstances, a secondary device is a non-medical device. In some circumstances, a secondary device (or a portion thereof) can be located proximal to the host. In some circumstances, a secondary device (or a portion thereof) can be located remotely from the host.

The terms “substantial” and “substantially” as used herein are broad terms, and are to be given their ordinary and customary meaning to a person of ordinary skill in the art (and are not to be limited to a special or customized meaning), and refer without limitation to a sufficient amount that provides a desired function. For example, an amount greater than 50 percent, an amount greater than 60 percent, an amount greater than 70 percent, an amount greater than 80 percent, or an amount greater than 90 percent.

The term “titrate,” as used herein is broad term, and is to be given its ordinary and customary meaning to a person of ordinary skill in the art (and is not to be limited to a special or customized meaning), and refers without limitation to delivery (e.g., over time) of controlled amounts of a substance to a host until a predetermined endpoint is reached. In some circumstances, the substance is a medicament and the endpoint is a predetermined medicament concentration. In some circumstances, the endpoint relates to a physical attribute of the host and/or an effect of the medicament. In some circumstances, an appropriate medicament-dosing regimen/schedule/procedure can be determined by titration, taking into account the observed pharmacokinetic characteristics of the agent in the individual subject.

The term “therapeutic window,” as used herein is broad term, and is to be given its ordinary and customary meaning to a person of ordinary skill in the art (and is not to be limited to a special or customized meaning), and refers without limitation to an index for estimation of drug dosage which can treat disease effectively while staying within a safety range. For example, it is the dosage of a medication between the amount that gives an effect (effective dose) and the amount that gives more adverse effects than desired effects. In some embodiments, a medicament with a small therapeutic window can be administered with care and control, such as by frequently measuring blood concentration of the drug, since it may easily lose effects or gives adverse effects.

The term “parameter” as used herein is a broad term and is to be given its ordinary and customary meaning to a person of ordinary skill in the art (and is not to be limited to a special or customized meaning), and furthermore refers without limitation to any factor that defines a system and determines (or limits) its performance. In some circumstances, a parameter can include information related to a medicament (e.g., identity, concentration, effects), a host (e.g., identity, weight, age, physical condition), a desired output, and the like.

The term “comprising” as used herein is synonymous with “including,” “containing,” or “characterized by,” and is inclusive or open-ended and does not exclude additional, unrecited elements or method steps.

All numbers expressing quantities of ingredients, reaction conditions, and so forth used in the specification are to be understood as being modified in all instances by the term “about.” Accordingly, unless indicated to the contrary, the numerical parameters set forth herein are approximations that may vary depending upon the desired properties sought to be obtained. At the very least, and not as an attempt to limit the application of the doctrine of equivalents to the scope of any claims in any application claiming priority to the present application, each numerical parameter should be construed in light of the number of significant digits and ordinary rounding approaches.

### Overview

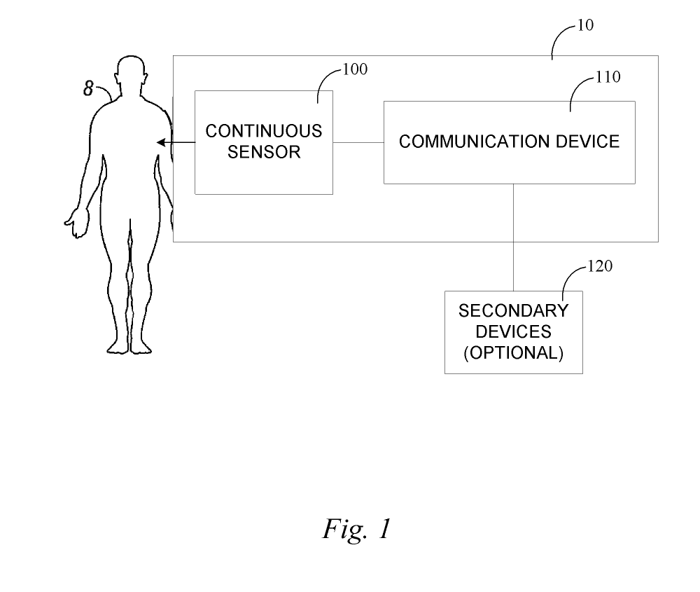

Referring to FIG. 1, the preferred embodiments provide a system 10 for the continuous detection of an analyte, wherein the system includes a continuous analyte sensor 100 and a communication device 110. In some embodiments, the system is configured to operably communicate with and/or integrate with a secondary device 120, which, depending upon the particular embodiment may or may not be a medical device. As is discussed herein, the system can be configured for use in a variety of settings and for a variety of purposes. Accordingly, in each aspect of the present system, the continuous analyte sensor and communication device are adapted for the unique demands of each unique setting/purpose. For example, in one aspect, the system is configured for drug titration in a medical setting. In another aspect, the system is configured for monitoring a host for the consumption of a drug/medicament, such as in a law-enforcement setting (e.g., monitoring for consumption of a drug of abuse) or a social work setting (e.g., monitoring for compliance with anti-tuberculosis therapy). In yet another aspect, the system is configured for monitoring a host's hormone levels, such as to predict ovulation, determine pregnancy and/or diagnose hormonal imbalances. In still another aspect, the system is configured for monitoring a host's nutritional status, such as in an intensive care, chronic care and/or post-operative setting. Additional aspects and embodiments of the present system are discussed herein.

### Continuous Medicament Titration System

One aspect provides a system configured for medicament titration in a host. Medicament titration may be conducted to determine an appropriate dosage of a medicament having a narrow therapeutic window. In some circumstances, the goal of medicament titration is to optimize the host's therapeutic response to the medicament while avoiding the medicament's adverse effects as much as possible. A medicament can be titrated, for example, by delivering defined amounts of the medicament to the host, while monitoring the medicament's concentration (e.g., in the plasma) and/or effects. The amount and timing of medicament delivery can be influenced by a variety of factors, including the host being treated (e.g., due to patient to patient variability), the severity of the affliction, the manner of medicament administration, the medicament's mechanism of action and/or pharmacokinetics, and the judgment of the prescribing physician. Medicament titration can be a slow and laborious process, requiring periodic collection of blood samples and laboratory testing thereof.

Continuous Medicament Sensor

FIG. 1 is a block diagram illustrating one embodiment of the continuous analyte sensor system 10, wherein the system is configured to provide information associated with a titration of a medicament, wherein the medicament is measured continuously in a host 8, including a continuous analyte sensor 100 and a communication device 110. A medicament, also referred to as a medicine or drug, is a substance or agent that is given to treat or prevent, alleviate the symptoms of disease and/or illness. Some medicaments, such as, for example, certain antibiotics, vasopressors and nitrovasodilators, heparin, coumadin, digoxygen, are known to have a narrow therapeutic window. In other words, there is a small safety cushion between a therapeutic dose and a toxic dose and/or between a therapeutic dose and a dose that produces certain side effects. Accordingly, in preferred embodiments, the system is configured and arranged for the determination of a medicament's therapeutic dose for a given host by titration of the medicament.

As a non-limiting example of medicament titration, heparin is a medicament prescribed to prevent blood clots, and, due to its powerful anticoagulant properties, must be carefully titrated as very small heparin doses can cause life-threatening bleeding in some circumstances. Heparin titration can be performed by repeated administration of small heparin doses with simultaneous monitoring the host's clotting time (e.g., the length of time it takes a given amount of blood to clot; as heparin doses increase, clotting time will also increase). Using such a titration procedure, the physician (e.g., the user) is better able to determine the largest possible heparin dose the host can tolerate without encountering bleeding problems. Accordingly, in some embodiments, the continuous analyte sensor 100 is configured to measure a signal associated with the concentration of heparin in the host 8. In other embodiments, the continuous analyte sensor 100 is configured to measure a signal associated with the concentration of other drugs having small therapeutic windows, such as but not limited to certain antibiotics, vasopressors and nitrovasodilators, coumadin, and digoxygen. In preferred embodiments, the continuous analyte sensor can be configured to measure a signal associated with any medicament in a host, in vivo.

Referring again to FIG. 1, in general, the preferred embodiments provide a continuous analyte sensor 100 that measures a concentration of a medicament of interest or a substance indicative of the concentration or presence of the medicament. In some embodiments, the analyte sensor is an invasive, minimally invasive, or non-invasive device, for example a subcutaneous, transdermal, intravascular, or extracorporeal device. In some embodiments, the analyte sensor can be configured to analyze a plurality of intermittent biological samples. The analyte sensor can be configured to use any method of analyte-measurement known in the art, including enzymatic, chemical, physical, electrochemical, immunochemical, spectrophotometric, polarimetric, calorimetric, radiometric, and the like.

In some embodiments, the analyte sensor 100 is a continuous electrochemical medicament sensor configured to provide at least one working electrode and at least one reference electrode, which are configured to measure a signal associated with a concentration of the analyte in the host, such as described in more detail herein. For example, in the case of a system for providing information associated with a titration of a medicament, the analyte sensor is configured to measure a signal associated with a concentration of the medicament in the host 8. The output signal is typically a raw data stream that is used to provide a useful value of the measured analyte concentration in a host to the patient or doctor, for example. However, the analyte sensors of some embodiments comprise at least one additional working electrode configured to measure at least one additional signal. For example, in some embodiments, the additional signal is associated with the baseline and/or sensitivity of the analyte sensor, thereby enabling monitoring of baseline and/or sensitivity changes that may occur over time. In some embodiments, the additional signal is associated with the concentration of another analyte (e.g., other than the medicament being titrated). In some embodiments, the analyte sensor is configured to measure two or more analytes, such as but not limited to two or more medicaments, a medicament and glucose, or a medicament and an analyte indicative of the medicament's effect on the host.

A wide variety of suitable detection methods are compatible with the preferred embodiments. For example enzymatic, chemical, physical, electrochemical, immunochemical, optical, radiometric, calorimetric, protein binding, and microscale methods of detection, can be employed in the preferred embodiments, although any other techniques can be used in alternate embodiments. Additional description of analyte sensor configurations and detection methods can be found in U.S. Patent Publication No. US-2007-0213611-1, U.S. Patent Publication No. US-2007-0027385-1, U.S. Patent Publication No. US-2005-0143635-1, U.S. Patent Publication No. US-2007-0020641-1, U.S. Patent Publication No. US-2007-0020641-1, U.S. Patent Publication No. US-2005-0196820-1, U.S. Pat. No. 5,517,313, U.S. Pat. No. 5,512,246, U.S. Pat. No. 6,400,974, U.S. Pat. No. 6,711,423, U.S. Pat. No. 7,308,292, U.S. Pat. No. 7,303,875, U.S. Pat. No. 7,289,836, U.S. Pat. No. 7,289,204, U.S. Pat. No. 5,156,972, U.S. Pat. No. 6,528,318, U.S. Pat. No. 5,738,992, U.S. Pat. No. 5,631,170, U.S. Pat. No. 5,114,859, U.S. Pat. No. 7,273,633, U.S. Pat. No. 7,247,443 U.S. Pat. No. 6,007,775, U.S. Pat. No. 7,074,610, U.S. Pat. No. 6,846,654, U.S. Pat. No. 7,288,368, U.S. Pat. No. 7,291,496, U.S. Pat. No. 5,466,348, U.S. Pat. No. 7,062,385 U.S. Pat. No. 7,244,582, U.S. Pat. No. 7,211,439, U.S. Pat. No. 7,214,190, U.S. Pat. No. 7,171,312, U.S. Pat. No. 7,135,342, U.S. Pat. No. 7,041,209, U.S. Pat. No. 7,061,593, U.S. Pat. No. 6,854,317, U.S. Pat. No. 7,315,752, and U.S. Pat. No. 7,312,040, each of which is incorporated herein by reference, in its entirety.

Communication Device

As shown in FIG. 1, in preferred embodiments, the sensor system 10 includes a communication device 110 that is configured to output information associated with titration of the medicament in the host 8 (e.g., titration information). The communication device is operably connected to the continuous medicament sensor 100 and optionally to a secondary device 120. As discussed herein, in some circumstances, the secondary device 120 is a medical device (also referred to herein as a secondary medical device). In some circumstances, the secondary device 120 is a non-medical device.

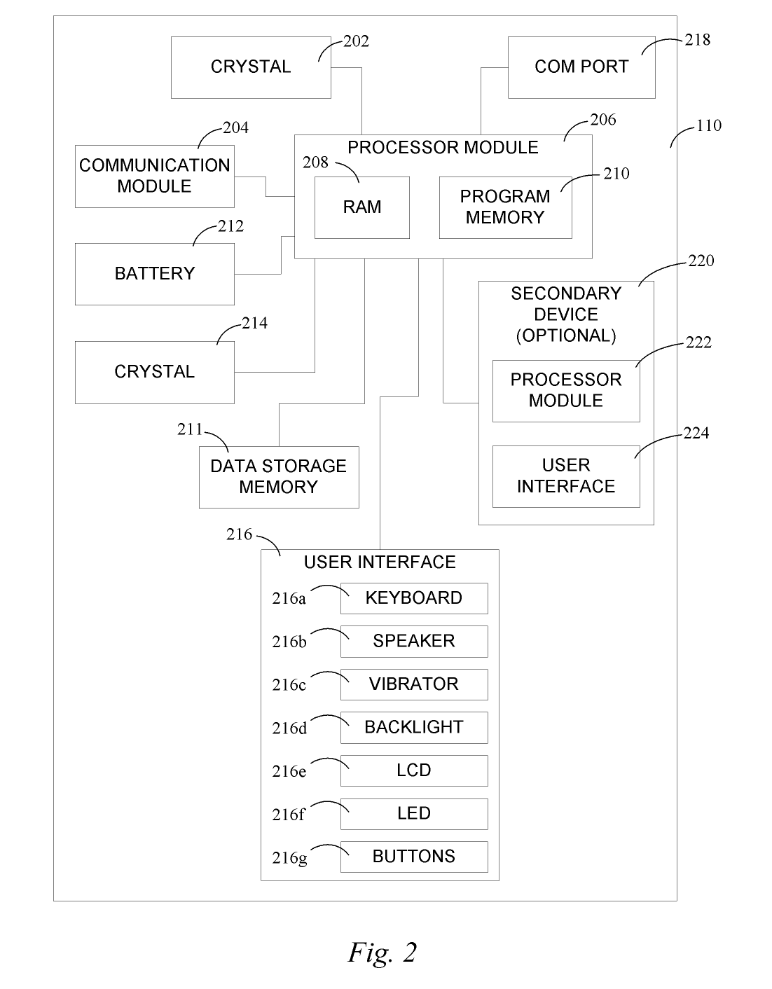

In preferred embodiments of a system 10 configured to provide information associated with a titration of a medicament, the communication device 110 includes an input module configured to receive titration parameters, which can be input via a user interface 216. Titration parameters can include any information needed to perform the medicament titration, such as but not limited to information related to the identity of medicament to be titrated (e.g., medicament name, original concentration), the target medicament concentration, a medicament concentration limit (e.g., a maximum and/or minimum acceptable concentrations, the maximum value of the therapeutic window), a toxic medicament concentration (e.g., including when alarms are to be provided), a medicament delivery rate, a medicament delivery time, host data (e.g., identification, height, weight, age, sex, a physical aspect/attribute of the host), and medicament effect information (e.g., a desired effect to be achieved, an adverse effect to be avoided, an effect to be detected). In some embodiments, a medical care provider (e.g., physician, nurse, technician) enters at least some of the titration parameters into the system 10, such as using the user interface 216, which is described with reference to FIG. 2. In some embodiments, the system is configured to receive at least some titration parameters from a secondary device and/or to intelligently calculate at least some of the titration parameters itself (e.g., updated titration parameters using data received and/or calculated).

In some embodiments, at least some titration parameters are programmed/entered/pre-set by the manufacturer. In some embodiments, the titration parameters are configurable, such as by the user (e.g., physician, technician, nurse). In preferred embodiments, the communications device 110 includes a processor module 206 (see FIG. 2) configured to process the signal from the continuous analyte sensor 100 and the titration parameters, to obtain titration information associated with titration of the medicament. In preferred embodiments, the system 10 includes electronics, also referred to as a “computer system” that can include hardware, firmware, and/or software that enable measurement and processing of data associated with analyte levels in the host. Portions of the electronics associated with the communication device are configured to receive and process sensor data and providing an output of medicament information (including storing information), and can reside on the sensor, a housing located adjacent to the sensor, on a vascular access device (and tubing and/or components connected thereto), on a bedside device, and/or on a remote device located remotely from the host's physical location, such as at a nurse's station, a doctor's office, a clinical lab, a medical records department and the like. In one exemplary embodiment, the electronics include a potentiostat (e.g., single and/or multi-channel), a power source for providing power to the sensor, and other components useful for signal processing. In another exemplary embodiment, the electronics include an RF module for transmitting data from sensor electronics to a receiver remote from the sensor. In another exemplary embodiment, the sensor electronics are wired to a receiver, which records the data and optionally transmits the data to a remote location, such as but not limited to a nurse's station, for tracking the host's progress and to alarm the staff if a therapy is required. In some embodiments, the output is to a secondary medical device. In some embodiments, the communication device is further configured to receive data and/or information from a secondary medical device and to optionally process the data and/or information. In some embodiments, the output includes instructions for a secondary medical device. In various embodiments, the communication device comprises at least a portion of sensor electronics and/or a processor module.

FIG. 2 is a block diagram that illustrates some of the electronics/components of the communication device 110 of the sensor system 10, which includes the electronics necessary for running the sensor 100, collecting and processing data, and outputting the titration information. Components of the communication device can be disposed on or proximal to the sensor, such as but not limited to located on/within a sensor housing. In some embodiment, wherein the sensor is configured for insertion into the host's circulatory system (e.g., a vein or artery), components of the communication device can be disposed on a vascular access device (e.g., a catheter or cannula) used to insert the sensor into the host, on a connector configured to couple the vascular access device to tubing, tubing to tubing, tubing to a fluid container, on a valve, and the like. In some embodiments, wherein the sensor is configured for transcutaneous insertion into the host, some or all of the electronics can be located in the sensor housing. In some embodiments, only a portion of the electronics (e.g., the potentiostat) is disposed on the sensor (e.g., proximal to the sensor), while the remaining electronics are disposed remotely from the sensor, such as on a stand or by the bedside. In a further embodiment, a portion of the electronics can be disposed in a central location, such as a nurse's station or clinic.

In additional embodiments, some or all of the electronics can be in wired or wireless communication with the sensor 100 and/or other portions of the communication device 110 and/or a secondary device 120. For example, a potentiostat disposed on the sensor and/or sensor housing can be wired to the remaining electronics (e.g., a processor module 206, a communication module 204, a recorder, a transceiver, etc.), which reside on the bedside. In another example, some portion of the electronics is wirelessly connected to another portion of the electronics, such as by infrared (IR) or RF. In one embodiment, a potentiostat resides on a tubing connector and/or valve and is connected to a receiver by RF; accordingly, a battery, RF transmitter, and/or other minimally necessary electronics are provided with the tubing connector and/or valve and the receiver includes an RF transceiver.

A battery 212 can be operably connected to the communication device 110 and provide the power for the sensor 100 or to another system component. In one embodiment, the battery is a lithium manganese dioxide battery; however, any appropriately sized and powered battery can be used (for example, AAA, nickel-cadmium, zinc-carbon, alkaline, lithium, nickel-metal hydride, lithium-ion, zinc-air, zinc-mercury oxide, silver-zinc, and/or hermetically-sealed). In some embodiments, the battery is rechargeable, and/or a plurality of batteries can be used to power the system. In some embodiments, a quartz crystal 214 is operably connected to the processor module 206 and maintains system time for the computer system as a whole, for example for the programmable acquisition time within the processor module. Alternatively, the system can be configured to plug into an electrical outlet.

A communication module 204 can be operably connected to the processor module 206 and transmits the sensor data from the sensor 100 to a receiver via a wired or wireless transmission. In some embodiments, mechanisms, such as RF telemetry, optical, infrared radiation (IR), ultrasonic, or the like, can be used to transmit and/or receive data.

Typically, the electronics include a processor module 206 that includes a central control unit that controls the processing of the sensor system 10. In some embodiments, the processor module includes a microprocessor, however a computer system other than a processor can be used to process data as described herein, for example an ASIC can be used for some or all of the sensor's central processing. For example, in some embodiments, the system is configured with an ASIC, wherein the ASIC includes at least RAM, programming memory and data storage memory (not shown). In some embodiments, the processor module typically provides semi-permanent storage of data, for example, storing data such as sensor identifier (ID) and programming to process data streams (for example, programming for data smoothing and/or replacement of signal artifacts such as is described in U.S. Patent Publication No. US-2005-0043598-1). The processor module additionally can be used for the system's cache memory, for example for temporarily storing recent sensor data. In some embodiments, the processor module comprises memory storage components such as program memory 208 (e.g., code for running algorithms), RAM 210, dynamic-RAM, static-RAM, non-static RAM, rewritable ROMs, non-volatile memory (e.g., EEPROM, flash memory, etc.), and the like.

In some embodiments, the processor module 206 comprises a digital filter, for example, an infinite impulse response (IIR) or finite impulse response (FIR) filter, configured to smooth the raw data stream from the A/D converter. Generally, digital filters are programmed to filter data sampled at a predetermined time interval (also referred to as a sample rate). In some embodiments, wherein the potentiostat is configured to continuously measure the analyte, for example, using a current-to-frequency converter, the processor module can be programmed to request a digital value from the A/D converter at a predetermined time interval, also referred to as the acquisition time. In these alternative embodiments, the values obtained by the processor are advantageously averaged over the acquisition time due the continuity of the current measurement.

In some embodiments, the processor further performs the processing, such as storing data (e.g., using data storage memory 211), analyzing data streams, calibrating analyte sensor data, estimating analyte values, comparing estimated analyte values with time corresponding measured analyte values, analyzing a variation of estimated analyte values, downloading data, and controlling the user interface by providing analyte values, prompts, messages, warnings, alarms, and the like. In such cases, the processor includes hardware and software that performs the processing described herein, for example flash memory provides permanent or semi-permanent storage of data, storing data such as sensor ID, and programming to process data streams (for example, programming for performing estimation and other algorithms described elsewhere herein) and random access memory (RAM) stores the system's cache memory and is helpful in data processing. Alternatively, some portion of the data processing (such as described with reference to the processor elsewhere herein) can be accomplished at another (e.g., remote) processor and can be configured to be in wired or wireless connection therewith.

In preferred embodiments, the communication device 110 includes an output module, which is integral with and/or operatively connected with the processor 206, and includes programming for generating output based on the data stream received from the sensor system and it's processing incurred in the processor. In preferred embodiments, output is generated via a user interface 216 configured to display the titration information. In preferred embodiments, the user interface 216 is configured to output the titration information and/or receive titration parameters.

In some embodiments, the user interface 216 is provided integral with (e.g., on the patient inserted medical device), proximal to (e.g., a receiver near the medical device including bedside or on a stand), or remote from (e.g., at a central station such as a nurse's station) the sensor electronics, wherein the user interface includes a keyboard 216a, a speaker 216b, a vibrator 216c, a backlight 216d, an LCD 216e or one or more LEDs 216f, and/or one or more buttons 216g. For example, in some embodiments, some of the user interface components can be proximal to the sensor, while other components of the user interface can be located remotely from the host. For example, a user interface including a display and buttons can be located on sensor housing or at the bedside while a second display and a speaker are located at the nurse's station. The components that comprise the user interface 216 include controls to allow interaction of the user (e.g., the medical personnel) with the sensor system 10. The keyboard can allow, for example, input of user information, such as mealtime, exercise, medicament administration, customized therapy recommendations, and reference analyte values. The speaker can produce, for example, audible signals or alerts for conditions such as approaching a toxic medicament concentration and/or the occurrence of an adverse effect of the medicament. The vibrator can provide, for example, tactile signals or alerts for reasons such as described with reference to the speaker, above. The backlight can be provided, for example, to aid a user in reading the LCD in low light conditions. The LCD can be provided, for example, to provide the user with visual data output. In some embodiments, the LCD is a touch-activated screen, enabling each selection by a user, for example, from a menu on the screen. The buttons can provide for toggle, menu selection, option selection, mode selection, and reset, for example. In some alternative embodiments, a microphone can be provided to allow for voice-activated control.

In some embodiments, prompts or messages are displayed on the user interface 216 to convey information to the user (e.g., the medical personnel), such as current medicament concentration, graphs of medicament concentration over time, current and/or predicted host status and/or level, current titration parameters, therapy recommendations, instructions, deviation of the measured analyte values from the estimated analyte values, alarms, and the like. Additionally, prompts can be displayed to guide the user through calibration, trouble-shooting of the calibration, and integration with a secondary medical device 120, selection of a medicament delivery and/or therapy protocol/parameters.

Additionally, data output from the communications device can provide wired or wireless, one- or two-way communication between the user interface and a secondary medical device 120 (sometimes referred to as an external device or a secondary device). In some embodiments, the system 10 is configured to display titration information on a secondary device, such as but not limited to a secondary medical device (e.g., on the user interface of the secondary medical device). In some embodiments, the system 10 is configured to display secondary medical device data/information (e.g., data/information from the secondary medical device) on the system's user interface 216. The secondary medical device can be any device that interfaces or communicates with the sensor system 10, such as via wired or wireless communication. In some embodiments, the secondary medical device is a computer, and the system 10 is able to download historical data for retrospective analysis by a nurse or physician, for example. In some embodiments, the secondary medical device is a modem or other telecommunications station, and the system is able to send alerts, warnings, emergency messages, and the like, via telecommunication lines to a party remote from the host, such as a user (e.g., a physician or other care provider). In some embodiments, the secondary medical device is a fluid delivery system, such as but not limited to a medicament pump, and the system 10 is configured to communicate therapy recommendations, such as medicament amount and time, to the pump. The secondary medical device can include other technology or medical devices, for example pacemakers, implanted analyte sensor patches, other infusion devices, telemetry devices, patient monitors, and the like. In some embodiments, the communications device includes a component of a secondary medical device.

The user interface 216, including keyboard, buttons, a microphone (not shown), and optionally the external device, can be configured to allow input of data. Data input can be helpful in obtaining information about the host (for example, host ID, age, sex, weight meal time, medicament administration, respiration, function of the heart and the like), receiving instructions from a physician (for example, procedural parameters, customized therapy recommendations, targets, criteria, thresholds, and the like), receiving calibration information, and downloading software updates, for example. Keyboard, buttons, touch-screen, and microphone are all examples of mechanisms by which a user (e.g., medical personnel) can input data directly into the system. A server, personal computer, personal digital assistant, medicament pump, and insulin pen are examples of external devices that can provide useful information to the receiver. Other devices internal or external to the sensor that measure other aspects of a patient's body (for example, temperature sensor, accelerometer, heart rate monitor, oxygen monitor, and the like) can be used to provide input helpful in data processing. In one embodiment, the user interface 216 can prompt the medical personnel to select an activity most closely related to the host's present activity, such as medication taken, surgical procedures, and the like, which can be helpful in linking to an individual's physiological patterns, or other data processing. While a few examples of data input have been provided here, a variety of information can be input, which can be helpful in data processing.

In one exemplary embodiment, the system is configured with one or more user-selectable/user-definable formats for the titration information output, such that the medical personnel can direct the system to output the titration information in one or more useful formats, such as by selection using a keyboard, a scroll menu or one or more dedicated buttons. In some exemplary embodiments, the system is configured with one or more locations for output, such that the medical personnel select one or more locations where the titration information is to be output, such as but not limited to at the host's bedside and/or at a remote location, such as a nurse's station, the doctor's office, a clinical laboratory or medical records. Advantageously, configuring the system for titration information output at remote locations enables medical personnel to monitor and/or review the host's past, present and predicted host status, including the host's current and historic titration information, without actually being in the room with the host. Similarly, in some embodiments, the system is configured with user selectable or user-definable information output (e.g., content), such that the medical personnel can select which titration information to output (e.g., concentration, change in concentration, and the like), for example.

Referring again to FIG. 1, in some embodiments, the system is configured to include a secondary medical device 120. In some embodiments, the communication device 110 is configured to receive information (e.g., data) from the secondary medical device. In some embodiments, the system is configured to output information to the secondary medical device. Any type of secondary medical device can be included in the system, depending upon the context of the system's use (e.g., cardiac ICU versus step-down ward), the system's particular configuration and the medicament to be titrated. In some embodiments, the secondary medical device includes at least one of a secondary analyte sensor and/or a patient monitor, and is configured to detect a second signal associated with an effect of a delivered medicament. In preferred embodiments, the effect of the delivered medicament is associated with a change in a host physical attribute, such as but not limited to blood pressure, heart rate, blood clotting rate, sedimentation rate, respiration rate, temperature, blood pH, levels of certain blood components, pain, response to nerve stimulation, concentrations of markers of inflammation or infection, changes in certain metabolites (e.g., urea, creatinine, etc.) and the like, and the secondary medical device is configured to detect this change. In some embodiments, the effect is associated with a metabolite related to the medicament and the secondary medical device is configured to detect this metabolite. In some embodiments, the secondary medical device is a medicament delivery device.

As a non-limiting example, wherein the medicament is a vasodilator (e.g., sometimes prescribed to heart attack and stroke patients to lower blood pressure), the secondary medical device can be an infusion pump (e.g., configured to deliver the vasodilator) or a blood pressure monitor (e.g., an intra-arterial blood pressure monitor) configured to monitor changes in the host's blood pressure (e.g., that occur during infusion of the vasodilator).

In some embodiments, the processor module is configured to determine an optimal dose of the medicament being titrated. What constitutes an “optimal dose” will depend upon the host, the medicament and the user (e.g., the physician). For example, in the case of a vasodilator, an optimal dose might be a blood concentration sufficient to maintain the host's blood pressure within a clinically acceptable window of blood pressures. Thus, in an exemplary embodiment, the system is configured to intelligently calculate and deliver optimal doses of the vasodilator to the host, such that the host is maintained within the window of blood pressures, as measured by the blood pressure monitor.

As a non-limiting example, in one embodiment, the medicament is IV vancomycin and the system is configured to provide information related to titration of the vancomycin. IV vancomycin is an antibiotic for the treatment of serious, life-threatening infections (by Gram-positive bacteria) that are unresponsive to other less toxic antibiotics. IV vancomycin has severe, possibly life threatening side effects. Accordingly, the system includes a continuous vancomycin sensor configured to measure the host's vancomycin concentration and an infusion pump configured to deliver doses of vancomycin as instructed by the system and/or the user. Accordingly, in this embodiment, the pump delivers the vancomycin doses to the host and the vancomycin sensor continuously detects a signal associated with the host's vancomycin concentration. Vancomycin titration parameters can be entered into the system, such as prior to delivery of the medicament to the host, and new (e.g., updated) parameters can be entered into the system (e.g., via the user interface) and/or intelligently calculated by the systems processor module. Titration information is output, such as via the user interface. In some embodiments, the system is configured to intelligently determine the optimal vancomycin dose (or a window of doses). In a further embodiment, after the optimal dose is determined, the system is configured to maintain the host's vancomycin concentration substantially at that level, such as via continuously monitoring the host's vancomycin level and controlling vancomycin delivery via the Infusion pump.

In some embodiments, the system is configured to operably connect to a secondary medical device configured to measure/detect a signal associated with an effect of the medicament (e.g., the medicament delivered to the host). In preferred embodiments, the input module is configured to receive the signal associated with the effect of the medicament. Additionally, in this embodiment, the processor module 206 is configured to process the signal associated with the concentration of the medicament (e.g., a first signal), the signal associated with the effect of the medicament (e.g., a second signal) and the titration parameters to obtain the titration information.

In still another example, the system is configured to titrate an appropriate oral dose of coumadin (e.g., an anticoagulant sometimes prescribed for blood clots and illnesses associated therewith), the system is configured to direct oral consumption of small amounts of coumadin, and includes a continuous coumadin sensor configured to measure the host's coumadin concentration and a secondary medical device configured to measure the host's clotting time (e.g., bleeding time).

In one exemplary embodiment, the system is configured and arranged for titration of vasodilating medicament to a host in need thereof (e.g., vasodilators are given to some heart attack and stroke patients, such as to lower blood pressure) and includes 1) a continuous analyte sensor configured to measure a first signal associated with the concentration of the vasodilator in the host, and 2) a communication device configured to receive and process data (e.g., via a processor module) from the analyte sensor as well as data received from one or more integrated/connected secondary medical devices, and to provide an output including titration information. For example, in this embodiment, the system is in operational communication with an intra-arterial blood pressure monitor, which is configured to measure a second signal associated with the host's blood pressure and to deliver the second signal (e.g., blood pressure data) to the communication device (e.g., via an input module) for processing with the first signal (e.g., a signal detected by analyte sensor) by the processor module. The system is also in operational communication with a medicament delivery device, such as a pump configured to deliver small amounts of the medicament (e.g., the vasodilator) to the host over time, wherein the changes in host blood pressure are substantially an effect of the delivered vasodilator. Accordingly, as the vasodilator is delivered to the host, the analyte sensor measures the vasodilator's concentration in the host (first signal) and the blood pressure monitor measures the host's blood pressure (second signal). The processor module received and processes the first and second signals with titration parameters to provide information related to the titration, such as but not limited to the relationship between the vasodilator's concentration and the host's blood pressure. In response to the host's blood pressure, the processor module is further configured to provide one or more instructions to the pump, in order to control the amount of vasodilator delivered to the host, in some embodiments. For example, if the host's blood pressure falls to an undesirably low level, the system is configured to instruct the pump to provide less medication. Similarly, if the host' blood pressure is measured to be above a preferred range, the processor is configured to provide an instruction to the pump to deliver the medication at an increased rate.

In some embodiments, the communication device is configured to provide one or more alarms. For example, in some embodiments, the processor module is configured to provide an alarm when the medicament concentration is substantially within a predetermined percentage of a medicament concentration limit. For example, the processor can be configured to provide an alarm when the host's plasma concentration of the drug is within 25%, 20%, 15%, 10%, and 5% of a toxic dose. In another example, the processor can be configured to provide an alarm when the medicament concentration is within a predetermined lower limit, such as the lowest dose of medicament that can be delivered.

In some embodiments, an alarm is visual (e.g., illumination and/or blinking of a light, transmission of a message to a display such as a screen), auditory (e.g., a buzzer or bell, transmission to an auditory device such as a telephone), vibratory (a portion of the system shakes, such as is used with pagers and cellular telephones), or combinations thereof. In some embodiments, a plurality of alarms can be used, wherein each alarm is related to a different host condition and/or event. For example, a first alarm can be associated with a first condition, and a second alarm can be associated with a second condition. In some embodiments, an alarm is associated with a particular event, such as but not limited to passage of a threshold, the presence of a selected compound, changes in vital signs, EEG changes, and the like.

In some embodiments, the system is configured mitigate drug-dosing errors. Accordingly, in some embodiment, the system is configured to monitor the host for the presence of a compound contraindicated for the host, and to provide an alarm and/or fail-safe if the contraindicated compound is detected in the host. For example, some hosts are subject to heparin-induced thrombocytopenia, and should not receive any fluids and/or medications containing heparin. Accordingly, in some embodiments, the system can be configured to detect heparin and to sound an alarm if and/or when heparin is detected. In another example, some hosts are allergic to one or more medicaments (e.g., aspirin, some antibiotics, etc.) or a compound used in the formulation of some medicaments (e.g., preservative or buffer components). Accordingly, the system can be configured to detect if the compound to which the host is allergic is detected, and to sound an alarm in the event of detection. In a further embodiment, the system is configured to deliver a counter-acting agent and/or resuscitating medicament to the host, such as epinephrine, or potassium sulfate.

In some embodiments, the system is configured with a library of medicaments from which the user can select. In some embodiments, the library includes one or more protocols and/or titration parameters associated with one or more of the medicaments in the library. In some embodiments, the user can select a medicament from the library (e.g., using the user interface), as well as select a protocol and/or one or more parameters related to the selected medicament, such as from a list thereof. In some embodiments, the system is configured such that the user can cancel, override and/or reprogram a protocol and/or parameter. In some embodiments, the system is configured to function with a plurality of interchangeable sensors, to intelligently detect the type of sensor to which it is connected, and optionally to present the protocol(s) and/or parameters related to the connected sensor type. For example, if the system is configured to work with a glucose sensor, an aspirin sensor or a heparin sensor, when the system is connected to a glucose sensor (e.g., by a user), the system is configured to intelligently determine that it is connected to a glucose sensor (e.g., not an aspirin or heparin sensor), and to optionally present preprogrammed glucose protocols, parameters and limits to the user for selection therefrom. The user can select a protocol, a parameter and/or a limit, and/or the user can cancel, override, and/or reprogram a protocol, a parameter and/or a limit.

Titration Information

As described elsewhere herein, the continuous analyte sensor is configured to continuously measure a concentration of a medicament in vivo and to provide a signal associated therewith. The communication device processes the signal to obtain titration information and to output that titration information. The data/signal can be processed, such as by the processor, to provide output and/or display the titration information. In some embodiments, the system is configured to receive and process data and/or information from a second medical device, and to use/output these data/information in conjunction with the titration information. In preferred embodiments, titration information can include any output information that is generated by the system. In some embodiments, the titration information includes at least one of a current medicament concentration, a predicted medicament concentration, a change in medicament concentration, an acceleration of medicament concentration, relationship of medicament concentration and a medicament concentration limit, an optimal medicament dose, rate of change information, a medicament clearance rate, and a correlation between a medicament concentration and an effect of the medicament (e.g., experienced by the host). In some embodiments, the titration information includes a therapy recommendation and/or a therapy instruction. In some embodiments, the recommendation/instruction is directed to a user (e.g., medical personnel) and directs the user to perform an action/task. These recommendations/instructions can include an alarm. For example, if the host is experiencing a severe level of a side effect, the instruction could be an alarm that alerts the user to terminate the procedure and/or to give the host an antidote to the medicament (e.g., if too much coumadin is delivered and the clotting time is too long, vitamin K can be given). In some embodiments, the recommendation/instruction is directed to a secondary medical device. For example, if the medicament is being delivered by an Infusion pump, the system can intelligently instruct the pump to increase and/or decrease the rate of medicament delivery. In some embodiments, the titration information can be used to intelligently process incoming data from the continuous analyte sensor 100 and any secondary medical devices, such as to optimize medicament delivery/titration.

Method of Medicament Titration

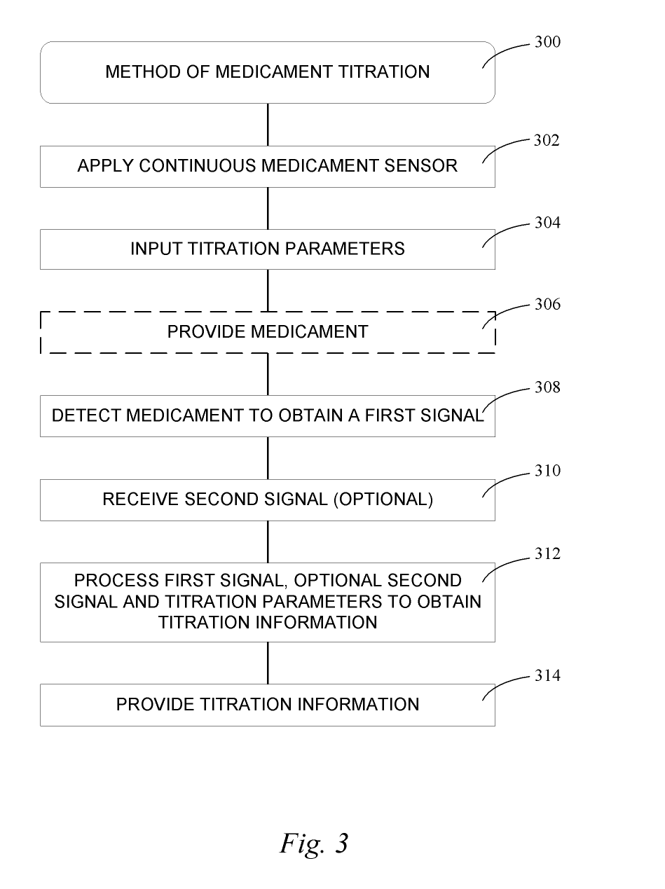

FIG. 3 is a flow chart 300 illustrating a method of medicament titration, in one embodiment. The medicament to be titrated can be any medicament. In some circumstances, the medicament is one having a narrow therapeutic window. In some circumstances, the medicament may be one that has severe side effects and the goal of titration is to determine the largest effective dose that the host can tolerate, while minimizing the side effects. Possible side effects can affect any part of the body and include (but are not limited to) diarrhea, nausea, alkaline phosphatase increase, rash, fever, headache, jaundice, vomiting, intermittent abdominal pain, gastritis, dyspepsia, muscle pain, nerve pain, somnolence, breathing difficulties, loss of taste, malaise, swelling/edema, confusion, dizziness, vertigo, foot drop, decrease in libido, depression, amnesia, tinnitus, asthenia, insomnia, bronchospasm, asthma, pharyngitis, rhinitis, sweating, conjunctivitis, and the like.

At block 302, a continuous medicament sensor 100 is applied to the host. In some embodiments, the sensor is configured for insertion/implantation in the host's circulatory system, and is inserted into a vein or artery via a catheter and/or cannula. Detailed descriptions of sensors configured for insertion into the circulatory system can be found in U.S. Patent Publication No. US-2008-0119703-1, U.S. Patent Publication No. US-2008-0119704-1, U.S. Patent Publication No. US-2008-0119706-1, U.S. Patent Publication No. US-2008-0108942-1, U.S. Patent Publication No. US-2008-0086042-1, U.S. Patent Publication No. US-2008-0086044-1, and U.S. Patent Publication No. US-2008-0086273-1, each of which is incorporated herein by reference in its entirety. In some embodiments, the sensor is configured for transcutaneous implantation into the host, such as but not limited through the skin of the abdomen. Additional description of transcutaneous insertion can be found in U.S. Patent Publication No. US-2006-0020187-1, which is incorporated herein by reference in its entirety. In some embodiments, the sensor is configured for extracorporeal application, such as an optical sensor configured to measure an analyte non-invasively, such as through the skin.

At block 304, titration parameters are input into the system 10, such as via the user interface 216. Titration parameters include, but are not limited to information related to how the titration procedure is to be performed. For example, titration parameters can include information related to the host, the medicament to be titrated, to the procedural steps to be followed, and the like. In another example, in some embodiments, the system is configured for entry of the medicament's identity, initial concentration and rate of delivery via a keyboard 216a or buttons 216g. In some embodiments, the system is configured for selection of host information (e.g., name, weight, age, height, etc.) via a scroll menu on an LCD screen 216e. Additional titration parameters can include a target medicament dose/concentration, a maximum and/or minimum concentration, and the like. In some embodiments, titration parameters can include a predetermined medicament concentration, or a percentage of a medicament concentration, which when reached an alarm is provided. For example, the processor module can be configured to provide the alarm when the medicament concentration is substantially within a predetermined percentage of a medicament concentration limit. For example, the predetermined percentage can be any percentage, such 5%, 10%, 15%, 20%, 25%, or more of a selected limit (e.g., the target concentration, a maximum or minimum concentration, at toxic dose, an amount/level of effect achieved, and the like). For example, in one embodiment, the communication device 110 is configured to provide an alarm when the host's plasma concentration is within 25% of the target concentration.

At optional block 306, the medicament to be titrated is provided to the host in a controlled amount, by any means known in the art, including orally, by injection and/or infusion, by inhalation, by absorption, and the like. In some embodiments, the system 10 is operably connected to and/or integrated with a secondary medical device 120 configured to deliver the medicament to the host, such as an infusion pump, for example (see FIG. 1). In some embodiments, the secondary medical device is configured to deliver the medicament at a predetermined, programmable and/or selectable rate. In some embodiments, the system is configured to provide instructions for medicament delivery to the secondary medical device. For example, in some embodiments, the processor is configured to evaluate the sensor information, the titration parameters, input information such as but not limited to information related to the medicament's effect, and the like, and to intelligently provide a therapy instruction to the secondary medical device.

At block 308, the system is configured to detect the medicament delivered (e.g., via the sensor) to obtain a signal. In some circumstances, the medicament delivery has just begun and the signal detected is a first signal. In some circumstances, an amount of the medicament has been delivered to the host, and the signal detected (e.g., via the sensor) is the current signal, which is related to the current medicament concentration. In preferred embodiments, the signal detected is related to the concentration of the medicament in the host; and can fluctuate, depending upon the medicament delivery rate and the rate of medicament clearance from the host's system/body.

At block 310, in some embodiments, the system 10 is optionally configured to receive a second signal. In some embodiments, a second sensor, such as a sensor configured to measure a signal associated with a second analyte, provides the second signal. A second sensor can be provided as a second analyte sensor integrated with the continuous medicament sensor 100 or as a separate device (e.g., a secondary medical device) that makes an operable connection with the system, such as by communicating with the communication device 110.

At block 312, the system is configured to process the first signal, an optional second signal and the titration parameters to obtain titration information. In preferred embodiments, the processor module 206 processes the signals and titration parameters. In preferred embodiments, the continuous analyte sensor 100 is continually providing data to the processor module. Accordingly, in preferred embodiments, the system is configured to at least intermittently process the data and provide updated titration information. For example, in some embodiments, the processor module is configured to process the incoming data every 5, 10, 15, or 30 minutes. In some embodiments, the processor module is configured to process the data every 1, 2 or 3-hours, or to wait even longer periods between processing. In some embodiments, the frequency with which the data are processed is a titration parameter that is entered/selected by the user. In some embodiments, the frequency of processing the data is dependent upon the length of time between medicament delivery and an effect of the delivered medicament can be detected.

At block 314, the system is configured to provide the titration information. In some embodiments, the titration information is provided via the user interface 216. For example, the current medicament concentration and the correlation between the medicament concentration and the medicament's effect can be displayed on a monitor at the host's bedside. In another example, titration information can be provided remotely from the host, such as at the nurse's station or in a senior physician's office. In some embodiments, the titration information can be displayed on the user interface of a secondary medical device, such as but not limited to a patient monitor or an Infusion pump. In some embodiments, titration information can be used as a titration parameter when the system performs subsequent processing of data being received.

Method of Multiple Sclerosis Medicament Titration

Multiple sclerosis (MS) is a chronic inflammatory demyelinating disease that affects the central nervous system, which controls many of the body's function. MS can be manifested in a variety of ways, including but not limited to muscle spasms and spasticity, muscle atrophy, pain, sensory dysfunction, cognitive dysfunction and brain atrophy, and loss of coordination/balance. While there is no known cure, there are many treatments (e.g., anti-multiple sclerosis medicaments) both for the modification of the disease progression and for the symptoms of multiple sclerosis. In some circumstances, some anti-multiple sclerosis medicaments may require titration to determine the optimal medicament dose. Accordingly, in some embodiments, the medicament is an anti-multiple sclerosis medicament and the effect of the delivered medicament is a change in at least one of a multiple sclerosis symptom and/or a side effect of the anti-multiple sclerosis medicament.

Method of Epilepsy Medicament Titration

Epilepsy is a group of common chronic neurological syndromes with vastly divergent symptoms characterized by recurrent unprovoked seizures, due to abnormal, excessive or synchronous neuronal activity in the brain. In many circumstances, epilepsy can be controlled, but not cured, with anti-epilepsy medications. Therapeutic doses (the dose at which seizures are controlled and side effects are minimal and/or tolerable) may vary widely among patients. For some patients, better seizure control can be reached by titrating an anti-epilepsy medicament, such as by measuring blood concentrations and correlating that with seizure occurrences, which can tailor a medication to suit an individual patient's specific and relatively variable body chemistry. In some circumstances, such as a seizure flurry, it can be useful to know if the serum medicament level is very high or very low. Accordingly, in some embodiments, the medicament is an anti-epilepsy medicament and the effect of the delivered medicament is a change in at least one of an epilepsy symptom and/or a side effect of the anti-epilepsy medicament.

Method of Anesthesia Medicament Titration

Anesthesia has traditionally meant the condition of having the feeling of pain and other sensations blocked. This allows patients to undergo surgery and other procedures without the distress and pain they would otherwise experience. Anesthesia medicaments provide one or more aspects of anesthesia, such as but not limited to analgesia, unconsciousness, amnesia, relaxation, and obtundation of reflexes. Various levels of anesthesia can be achieved, ranging on a continuum of depth of consciousness from minimal sedation to general anesthesia. The depth of consciousness of a patient may change from one minute to the next. Thus, many anesthesia medicaments are titrated to provide a desired level of unconsciousness. In some embodiments, the system is configured to provide information associated with titration of an anesthesia medicament. In some embodiments, the system includes a continuous analyte sensor configured to measure the level of medicament in the host. In some embodiments, the system is configured to operably connect to a secondary medical device. In preferred embodiments, the secondary medical device is an anesthesia device (e.g., a device used in the process of providing anesthesia to a host in need thereof). For example, in some embodiments, the anesthesia device is a patient monitor configured to monitor a host characteristic, such as but not limited to blood pressure, heart rate, respiration, oxygen saturation of the blood, neurological/brain function, muscle function, and the like. In another example, in some embodiments, the anesthesia device is configured to deliver the anesthesia to the host, such as but not limited in periodic and/or intermittent metered doses, or at a continuous rate.

### Continuous Ambulatory Drug Monitoring

One aspect provides a system for continuous monitoring of medicament consumption by an ambulatory host. For example, in some circumstances and/or in some settings, it is desirable to know if a host has taken a medicament, or not. In one exemplary circumstance, it can be desirable to continuously monitor a host for the consumption of a drug of abuse (DOA); if the host consumes the banned substance, law enforcement, correctional and/or medical personnel can respond as dictated by protocol and/or law. In another exemplary setting, it can be desirable to continuously monitor a host for compliance with a prescribed medicament dosing regimen, such as in the case of tuberculosis treatment; if the host's medicament blood concentration falls below a predetermined level, such as due to noncompliance with the prescribed therapy, the appropriate medical, social work, legal and/or law enforcement personnel can respond appropriately.

Ambulatory Host Monitor

In preferred embodiments, a system 10 for continuous ambulatory drug testing is provided, including an ambulatory host monitor, is provided. In preferred embodiments, the ambulatory host monitor is configured and arranged to provide information associated with the presence of a drug in a host, such as to monitor the host's consumption of at least one pre-selected medicament, and includes a continuous analyte sensor 100, a location module, a processor module, and a transmitter. The continuous analyte sensor can be transcutaneous, intravenous, wholly implantable or extracorporeal, and can use any method of detection known in the art, as described in the section entitled “Continuous Analyte Sensor.” The continuous analyte sensor 100 is configured to detect a signal associated with a presence of a drug in vivo. The drug that the sensor 100 is configured to detect can be any drug that the host can consume. In some circumstances, the drug is a prescribed medication, such as a treatment for an illness. For example, in some embodiments, the prescribed medication is an antibiotic prescribed for treatment of an infectious disease, and the continuous analyte sensor is configured to detect a signal associated with the concentration of the prescribed antibiotic in the host. Accordingly, in some embodiments, analyte sensor is configured to detect a signal associated with a drug that is a medicament, and the drug information includes information associated with a presence of the medicament in the host. In some circumstances, the drug is a drug that may be used by the host inappropriately or which may be habit forming; these drugs may be referred to as “drugs of abuse.” In some circumstances, such as law enforcement and/or drug rehabilitation settings, it is desirable to monitor a host/patient, such as to alert a caretaker if and/or when the host does consume the monitored substance. Accordingly, in some embodiments, the drug is a DOA and the continuous analyte sensor 100 is configured to detect a signal associated with the concentration of the DOA in the host, and the drug information includes information associated with the presence of the DOA in the host. DOAs are discussed in greater detail below.

As a non-limiting example, treatment of tuberculosis (TB) is one circumstance wherein the consumption of an antibiotic can be monitored. Compliance with TB antibiotic treatment is a major public health problem, since TB antibiotic therapy can take a few months to a year or longer to complete. In some circumstances, host/patient compliance is so problematic that the host is required to present himself daily, for medication, with live observation of the actual consumption. In some circumstances, the host/patient may even be jailed, to force compliance. If the host/patient does not comply with the prescribed treatment, the TB bacterium that is infecting him will likely become resistant to that antibiotic. The host (e.g., the patient) may experience infectious periods during which he can spread the disease to other people, which results in the spread of drug-resistant TB strains. The host's compliance with the drug therapy may be monitored/followed by a user (e.g., a caretaker, case-manager), such as but not limited to a physician, a nurse, a physician's assistant, a technician and/or a social worker. The most commonly prescribed antibiotics (anti-tuberculosis medicaments/drugs) include streptomycin, isoniazid, rifampicin, ethambutol, and pyrazinamide. However, a variety of other drugs may be tried, including aminoglycosides (e.g., amikacin, kanamycin), polypeptides (e.g., capreomycin, viomycin, enviomycin), fluoroquinolones (e.g., ciprofloxacin, moxifloxacin), thioamides (e.g. ethionamide, prothionamide), cycloserine, and p-aminosalicylic acid. In some circumstances, a drug regimen, including two or more antibiotics, may be prescribed. Accordingly, in some embodiments, the ambulatory host monitor is configured to continuously detect a signal associated with a concentration of an anti-tuberculosis medicament in a host in need there of. In one exemplary embodiment, analyte sensor is configured to detect a signal associated with a drug that is an anti-tuberculosis medicament, and the drug information includes information associated with a presence of the anti-TB medicament in the host. In some embodiments, the ambulatory host monitor is configured to detect two or more anti-TB medicaments in the host. In some embodiments, the ambulatory host monitor is configured to output information related to the concentration of the medicament in the host. In some embodiments, the ambulatory host monitor is configured to provide an alert, such as a visual, auditory and/or tactile alert, to the host, such as but not limited to a reminder to take the prescribed medicament that the sensor is monitoring. In some embodiments, the ambulatory host monitor is configured to provide information, an alert and/or an alarm, such as to a user, such as to notify the user of the host's compliance and/or lack thereof. A more detailed description of the use of the ambulatory host monitor can be found in the section entitled “Method of Continuous Ambulatory Drug Testing.”

As another non-limiting example, drug abuse rehabilitation (or in a sports or law enforcement setting) is another circumstance wherein medicament consumption may be monitored. DOAs and alcohol are the most frequent causes of driving under the influence, in addition to a host of other problems related to their use. For example, illegal drug use and excessive use of alcohol contribute to many accidents, injuries and medical conditions. Screening individuals for DOAs and alcohol is an important method in identifying those who may cause harm to themselves and others. Screening can also provide an additional benefit as a deterrent against inappropriate and/or illegal use of drugs or alcohol. Representative DOAs (including misused drugs), by way of example and not limitation, include (i) alkaloids such as morphine alkaloids, which include morphine, codeine, heroin, dextromethorphan, their derivatives and metabolites; cocaine alkaloids, which include cocaine and benzyl ecgonine, their derivatives and metabolites; ergot alkaloids, which include the diethylamide of lysergic acid; steroid alkaloids; iminazoyl alkaloids; quinazoline alkaloids; isoquinoline alkaloids; quinoline alkaloids, which include quinine and quinidine; diterpene alkaloids, their derivatives and metabolites; (ii) steroids, which include the estrogens, androgens, and reocortical steroids, bile acids, cardiotonic glycosides and aglycones, which includes digoxin and digoxigenin, saponins and sapogenins, their derivatives and metabolites; steroid mimetic substances, such as diethylstilbestrol; (iii) lactams having from 5 to 6 annular members, which include the barbiturates, e.g., Phenobarbital and secobarbital, diphenylhydantoin, primidone, ethosuximide, and their metabolites; (iv) aminoalkylbenzenes, with alkyl of from 2 to 3 carbon atoms, which include the amphetamines; catecholamines, which include ephedrine, L-dopa, epinephrine; narceine; papaverine; and metabolites of the above; (v) benzheterocyclics which include oxazepam, chlorpromazine, tegretol, their derivatives and metabolites, the heterocyclic rings being azepines, diazepines and phenothiazines; (vi) purines, which includes theophylline, caffeine, their metabolites and derivatives; (vii) drugs derived from marijuana, which include cannabinol and tetrahydrocannabinol; (viii) hormones such as thyroxine, cortisol, triiodothyronine, testosterone, estradiol, estrone, progesterone, (ix) tricyclic antidepressants, which include imipramine, dismethylimipramine, amitriptyline, nortriptyline, protriptyline, trimipramine, chlomipramine, doxepine, and desmethyldoxepin; and (x) anti-neoplastics, which include methotrexate; and the like. Accordingly, in some embodiments, the ambulatory host monitor is configured to continuously detect a signal associated with a concentration of a DOA in a host. In some embodiments, the ambulatory host monitor is configured to detect two or more DOAs in the host. In some embodiments, the ambulatory host monitor is configured to output information related to the concentration of the one or more DOAs in the host. In some embodiments, the ambulatory host monitor is configured to provide an alert, such as a visual, auditory and/or tactile alert, to the host. In some embodiments, the ambulatory host monitor is configured to provide information, an alert and/or an alarm, such as to a user (e.g., a caretaker, caseworker or law enforcement personnel), such as to notify the user of the host's consumption of a DOA (and/or lack thereof). A more detailed description of the use of the ambulatory host monitor can be found in the section entitled “Method of Continuous Ambulatory Drug Testing.”

The continuous analyte sensor 100 can be configured for invasive and/or noninvasive application to the host 8. For example, in some embodiments, the sensor is configured for transcutaneous application to the host, such as in the abdomen and/or a limb (e.g., arm or leg). In some embodiments, the sensor is configured for insertion into the host's circulatory system, such as via a catheter/cannula. In some embodiments, the sensor is configured for external application to the host, such as an optical sensor applied to the host's skin using an adhesive, straps and/or other attachment means. In some circumstances, a host may be tempted to tamper with the sensor or another portion of the ambulatory host monitor. Thus, in some embodiments, the system is configured to prevent the host from tampering therewith, such as by inclusion of blocking structures and/or locks, which prevent host access to the system and/or removal of the system and/or a signaling mechanism configured to alert the user in the event the host tampers with the system. In some circumstances, a wholly implantable sensor can be preferred, as the ability of the host to tamper with the device would be severely curtailed. In some embodiments, host tampering with the system can be detected due to changes in the sensor signal detected. For example, in some circumstances, an analyte sensor produces a continuous low level of signal (e.g., background noise). While sensors sometimes malfunction, in many circumstances, background noise termination can be attributed to host removal (and/or deactivation) of the ambulatory host monitor/analyte sensor. In some embodiments, the system is configured to provide an alarm if a portion of the sensor signal (e.g., noise) drops below a predetermined level.

In preferred embodiments, the ambulatory host monitor is configured to provide the sensor's location, in addition to the continuous analyte sensor. For example, in some circumstances, user monitoring the host may need to determine the host's location, such as in order to respond to information provided by the system. Accordingly, in preferred embodiments, the ambulatory host monitor includes a location module configured to provide a location of the continuous sensor. The location module can provide the location using a signal, such as via wireless communication. In some embodiments, the location module includes a Global Positioning System (GPS) module configured to determine the sensor's location using GPS. If the ambulatory host monitor is not removed and/or disabled by the host, then the location is indicative of the host's location. If the host removes and/or disabled the ambulatory host monitor, then there will be no signal associated with drug consumption and/or background noise. In some embodiments, the system is configured to provide an alert to the user (e.g., a caregiver and/or other monitoring personnel) if there is substantially no signal measured by the continuous analyte sensor.

In preferred embodiments, the ambulatory host monitor includes a processor module configured to process the signal from the continuous analyte sensor 100, to obtain drug information. The processing can be performed using any useful method and/or algorithm, such as but not limited to those described elsewhere herein. In some embodiments, the system is configured to store drug information in data storage memory. For example, the processor can process the signal and store the processed information in the data storage memory for future use in another analysis, such as to produce trend information. In general, drug information includes information related to the host's consumption of a selected DOA. For example, in some embodiments, the drug is cocaine and the drug information includes information such as the concentration of the cocaine in the host. In another exemplary embodiment, the drug is alcohol and the drug information includes information related the concentration of alcohol in the host. In still another exemplary embodiment, the drug is an anabolic steroid, and the drug information includes information related to the presence of the anabolic steroid in a host, such as a professional athlete. In some embodiments, drug information can include host identifying information, date and time, drug identity, current drug concentration, changes in drug concentration, rate of change information, trend information, and the like, which can also be stored in data storage memory for future use.

In some embodiments, the processor module is configured to provide an alarm when the signal (e.g., detected by the analyte sensor) is below (or above) a programmed level. For example, the system can be configured by the manufacturer to provide an alarm (e.g., transmitted to the caretaker) when the detected signal is substantially equal to and/or below (or above) a level of background noise. In some embodiments, the manufacturer can preprogram a plurality of alerts related to the signal detected, such that the user can select a signal level below/above which the alert is provided. Selection of the level could be accomplished using the user interface, such as via a pop-up menu on a screen/display operably connect the ambulatory host monitor, or by typing one or more commands/parameters into the user interface, such as via a keyboard. In some embodiments, the user interface is releasably connected to the ambulatory host monitor, such as when the ambulatory host monitor is being applied to the host, such as for input of parameters into the ambulatory host monitor. In some embodiments, the ambulatory host monitor is configured such that parameters can be input wirelessly.

In preferred embodiments, the ambulatory host monitor includes a transmitter configured to transmit the drug information, such as to a remote receiver (e.g., communication device), described in the section entitled “Remote Monitoring.” In preferred embodiments, the transmitter is configured to transmit the location provided by the location module. In some embodiments, the ambulatory host monitor is configured to transmit the information wirelessly, such as to a proximal receiver (e.g., located at the host's home) configured to receive the information from the ambulatory host monitor, wherein the proximal receiver then transmits the received information to a remotely located receiver (e.g., via either wired or wireless communication, such as the telephone or the Internet), such as but not limited to a central monitoring location or a caregiver's office. In some embodiments, the ambulatory host monitor is configured to transmit the information on a continuous and/or continual basis, such as every 10-30 minutes, every hour, every 2, 3, 4 or 5 hours, twice a day, and the like. In some embodiments, the ambulatory host monitor is configured such that the host must plug it into a secondary device for transmission of the information. For example, the system can be configured such that the host must regularly and/or periodically connect his ambulatory host monitor to a telephone or to a computer connected to the Internet to transmit the drug information/location to the user. In some embodiments, the ambulatory host monitor is configured to provide an alert (e.g., auditory, visible, tactile, etc.) to the host, such as a reminder to connect his ambulatory host monitor to the phone and to transmit the information.

In some circumstances, it can be desirable to test a host 8 for drug use prior to operation of heavy machinery or prior to entering a hazardous area (e.g., a factory, laboratory, or other work facility containing heavy machinery and/or hazardous substances), such that if the drug is detected in the host, the host will not be able to operate the machinery and/or enter the hazardous area. In an exemplary embodiment, the system includes a secondary device configured to operably connect with the ambulatory host monitor. The ambulatory host monitor is configured to provide drug information to the secondary device, wherein the secondary device is configured to provide an alert and/or to deactivate a machine. For example, in some embodiments, the secondary device is a receiver operably connected to the starting mechanism of an automobile. The host must initiate transfer of drug information to the secondary device, such as by engaging a wired and/or wireless connection between the ambulatory host monitor and the secondary device. The ambulatory host monitor can be configured to transfer the drug information to the secondary device, such as via an output module, and the secondary device can be configured to receive the drug information, such as via an input module. In some embodiments, the secondary device includes a processor module configured to process the drug information and to provide an instruction to the machinery to which it is operably connected (e.g., via a wired connection or wirelessly), depending upon the presence of the drug in the host. For example, if the drug information indicates that the host's concentration of the drug is above a predetermined level, then the secondary device is configured to instruct the machinery to not activate (e.g., prevents the machine from turning on). However, if the host's drug concentration is below a predetermined level, then the secondary device is configured to instruct the machinery to activate (e.g., turn on).

As of August 2005, it is illegal to drive with a blood alcohol content (BAC) of 0.08 or higher. While a BAC of 0.01-0.029 has only subtle effects on the host, a BAC of 0.03-0.059 can impair alertness, judgment and coordination. In some circumstances, it is desirable to prevent an intoxicated individual, such as a person previously convicted of driving under the influence (e.g., DUI, drunk driving). In some circumstances, this is done by connecting a blood alcohol Breathalyzer test to a car, such that the individual has to perform the Breathalyzer test and have a BAC below a predetermined level before the car will turn on. As a non-limiting example, the secondary device is configured to operably connect to an automobile and to allow or prevent the host from turning on (operating) the automobile, depending upon the amount of alcohol detected in the host (by the host's ambulatory host monitor). For example, the host can be required to connect his ambulatory host monitor (wired or wirelessly) to the secondary device. The secondary device receives and processes the drug information from the ambulatory host monitor. If the host's alcohol content is equivalent to and/or above a preprogrammed level (e.g., a BAC of 0.03, 0.04, 0.045, etc.), then the secondary device prevents the automobile from turning on, such as by deactivating the engine. If the host's drug information indicates a level below the preprogrammed level, then the secondary device allows the automobile to turn on.

In a related embodiment, the secondary device can be a device configured to allow and/or prevent the host from entering a specific area, depending upon his DOA/alcohol consumption. For example the secondary device can be installed at the entrance to a factory, a laboratory, and the like. In some embodiments, the secondary device can be operably connected to and/or integrated with an electronic time clock configured to record the times an employee begins/completes a work shift, such that the electronic time clock records the employee's blood alcohol level and/or prevents payment for work conducted when the host had a DOA/alcohol level above a predetermined level. In some embodiments, the secondary device is configured to provide an alert and/or an instruction, such as to alert a supervisor and/or to control the opening of a door to the work area.

Remote Monitoring

In some embodiments, the system 10 includes a communication device 110 located remotely from the ambulatory host monitor, such as but not limited to proximal to the user (e.g., personnel monitoring the host 8). For example, in the case of monitoring a TB patient for compliance with a treatment/therapy protocol, the communication device can be located at a doctor's office, in a clinic or hospital, at a social worker's office, or even a law enforcement facility. In the case of monitoring a host for drug abuse, the communication device might be located at a law enforcement facility, such as a correctional/parole officer's office, a police department, a judicial facility (e.g., associated with a court or judge's offices), at the offices of a drug rehabilitation facility, at the office of a social worker, and the like. In some embodiments, the communication device is configured as at least two parts, wherein one part is located proximal to the host and another part is located remotely from the host.

In preferred embodiments, the communication device 110 (and/or a secondary device) is configured to receive the drug information and the location (e.g., from the ambulatory host monitor), to process the drug information and the location to obtain drug-monitoring information, and to output the drug-monitoring information. Drug-monitoring information can include (but is not limited to) any information related to the host identity, the drug being monitored, consumption of the drug, and the location of the ambulatory host monitor. In some circumstances, a user can monitor a plurality of hosts simultaneously. Accordingly, in preferred embodiments, the communication device is configured to receive drug information and locations from a plurality of ambulatory host monitors (e.g., one for each host being monitored), to process the drug information and location from each ambulatory host monitor to produce drug-monitoring information for each host, and to output each host's drug-monitoring information. For example, in one embodiment, the system is configured such that a user can monitor three hosts, each being monitored for consumption of a different DOA. For example, host A can be monitored for drug #1, host B for drug #2, and host C for drug #3. Accordingly, in this embodiment, the communication device is configured to receive drug information from each host's ambulatory host monitor; the received drug information for each host can include host identification (e.g., A, B or C), the drug monitored (e.g., #1, #2 or #3), and each host's current drug concentration. In some embodiments, the ambulatory host monitor can be configured to transmit drug information only if the drug is measured in the host. If no drug is measured, then the system 10 can be configured to transmit drug information less frequently (e.g., once a day, such as to provide confirmation that the host is still wearing the ambulatory host monitor and/or information related to the device's function), or not at all. Conversely, if the sensor 100 detects a signal associated with the presence of the drug in the host, the system can be configured such that drug information related thereto is transmitted substantially immediately to the communication device 110.

In some embodiments, the drug-monitoring information includes an instruction and/or a recommendation. In an exemplary embodiment, the communication device is configured to instruct the user to interact with the host 8. For example, the system could instruct the user to call the host, to go to the host's location, or to instruct law enforcement personnel to arrest the host.

As a non-limiting example, in one embodiment, the system 10 is configured for use with competitive athletes, such as to screen for the use of banned performance-enhancing substances, such as but not limited to anabolic steroids and erythropoietin. Accordingly, the system is configured such that each of a plurality of athletes can wear an ambulatory host monitor (e.g., configured to detect one or more preselected/preprogrammed analytes/banned substances), wherein each of the ambulatory host monitors transmits its drug information to a communication device 110, wherein the communication device is configured to process the drug information from each ambulatory host monitor to provide drug-monitoring information related to banned substance (e.g., a steroid, erythropoietin or other drug) consumption via the athletes. The communication device can be configured to provide an alert and/or instruction to a user of the communication device, such as monitoring personnel and/or an event official.

In some embodiments, the system is configured to detect a presence of a medicament (or another substance) in the host and optionally the consumption of the medicament by the host. For example, many children with asthma are allowed to treat themselves with inhaled medications, such as but not limited to rapid/rescue inhaled steroids. However, the medication may appear to not be working. In some circumstances, the medication taken may not be working sufficiently to alleviate the child's symptoms, but in some other circumstances, the child may not be taking the medication properly (which appears that the drug isn't working). It can be difficult to distinguish between these two possibilities. This type of quandary can happen with other medicaments the host self-administers. Accordingly, in some embodiments, the system is configured to detect and/or measure the drug in the host and to monitor the drug delivery. For example, the system can be configured to note each time a child uses his inhaler and to measure the concentration of the inhaled medication in the child's system. A user (e.g., parent, physician, nurse, etc.) can review the collected data and determine either if the drug is being consumed properly but isn't working sufficiently, or if the drug isn't being taken properly, so there is an insufficient level of the drug in the child's system to be sufficiently effective.

Method of Continuous Ambulatory Drug Testing

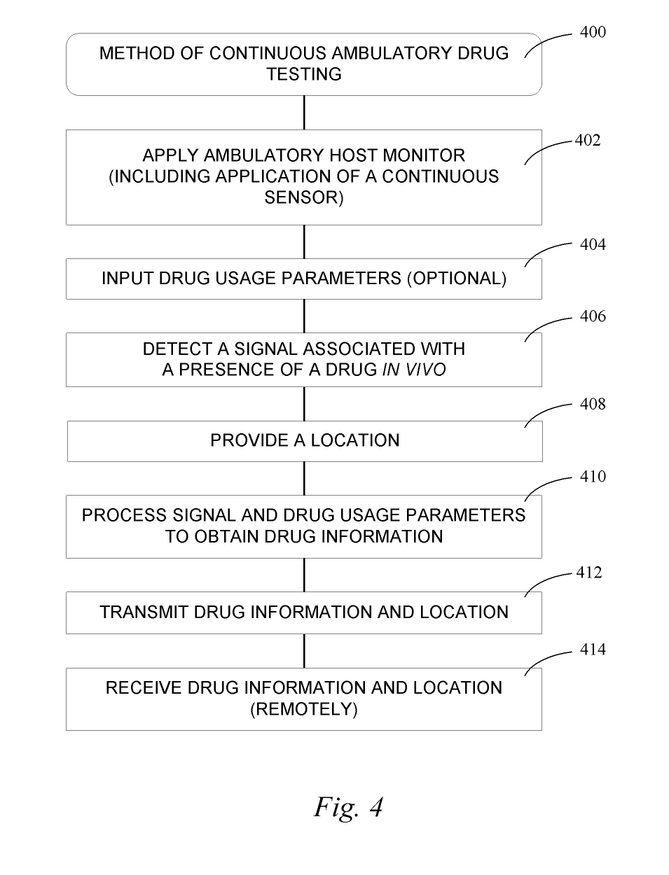

FIG. 4 is a flow chart 400 of a method of continuous ambulatory drug testing, in one embodiment.

At block 402, an ambulatory host monitor is applied to a host 8, such as a person to be monitored for consumption of the analyte detected by the sensor. The electronics associated with the sensor, including the locator module, processor module and transmitter must also be applied to the host. In some embodiments, the ambulatory host monitor is configured as a single unit configured to insert the sensor and to hold the electronics associated with the sensor. In some embodiments, the ambulatory host monitor is configured as two or more connectable units, such that the sensor can be inserted into the host, and then the unit containing electronics is connected to the sensor unit after sensor insertion. In some embodiments, the sensor unit is disposable while the second unit including the electronics is reusable. In some embodiments, the entire ambulatory host monitor is configured to be disposable. In some embodiments, the ambulatory host monitor includes a mechanism/structure configured to prevent tampering and/or removal of the device, such as by the host.

At block 404, drug usage parameters are optionally input into the ambulatory host monitor, such as by a user and/or the manufacturer. Drug usage parameters include but are not limited to information related to the host's identity, the identity of the drug to be detected, information related to limits (e.g., maximum concentration, minimum concentration, etc.) and information related to set points, such as for alarms and alerts, which information is to be transmitted to a remote communication device, the mode and time of transmission (e.g., via radio signal, which radio frequency, via telephone or Internet, whether or not the host will be required to connect the ambulatory host monitor to a secondary device for transmission of the information, etc.), information related to any secondary devices that are configured to connect/interact with the ambulatory host monitor, and the like. In some embodiments, the manufacturer configures the ambulatory test device to detect a specific analyte. For example, the ambulatory test device can be configured as an alcohol monitor and sold for that purpose only. In other embodiments, the ambulatory host monitor is configured to accept one or more of a variety of sensors. For example, in some embodiments, the sensors can be interchangeable and the electronics of the ambulatory host monitor are configured to receive & process a signal from any of those particular sensors. This configuration allows the user to select the analyte prior to application of the ambulatory host monitor to the host 8. In this embodiment, the user can select the analyte and/or sensor type from a menu, when applying the device to the host. In some embodiments, the sensor 100 is configured such that the electronics of the ambulatory host monitor can intelligently determine what kind of sensor it is (e.g., which drug the sensor is configured to detect). For example, a disposable sensor can include a physical key (e.g., RFID) and/or programming that can be detected by the device's electronics when the disposable sensor is installed in the ambulatory host monitor (e.g., prior to application of the device to the host). In some embodiments, the system is configured and arranged such that the ambulatory host monitor is operably connected to the communication device 110, for entry of the drug usage parameters, by either wired and/or wireless means of connection. This connection can be made prior to, during and/or after application of the device to the host. In some embodiments, the system is configured and arranged such that drug usage parameters can be transmitted to the ambulatory host monitor from a remote location. For example, a user at a location remote from the host can transmit parameters to the ambulatory host monitor attached to the host. In some embodiments, the ambulatory host monitor includes a user interface that can be used for entering drug usage parameters.

At block 406, a signal associated with a presence of the drug of interest in vivo is detected, such as by the analyte sensor of the ambulatory host monitor.

At block 408, a location of the ambulatory host monitor is provided, such as by the location module of the ambulatory host monitor. As described elsewhere herein, the location can be determined using a GPS tracking system. In circumstances wherein the ambulatory host monitor has not been removed from the host (or deactivated), the location of the ambulatory host monitor is substantially equivalent to the host's location. The provided location can be used to locate the host.

At block 410, the processor module of the ambulatory host monitor processes the signal (detected at block 406) and the drug usage parameters (optionally input at block 404 and/or input by the manufacturer) to obtain drug information. For example, in some embodiments, the system is configured to determine the concentration of the drug in the host and then to compare the drug concentration to the drug usage parameters, such as to determine if the concentration of the drug in the host exceeds a predetermined level.

At block 412, the transmitter of the ambulatory host monitor transmits the drug information and the location of the ambulatory host monitor.

At block 414, the drug information and location are received remotely, such as by a communication device located at a central facility, such as but not limited to an office of a user (e.g., a person charged with monitoring the host 8 for drug usage will operate the communication device 110).

In preferred embodiments, the communication device (e.g., a processor module component thereof) processes the received drug information and location to provide drug-monitoring information, which can be output via a user interface. Depending upon the desired output, the communication device can be configured to continuous and/or intermittently output the drug-monitoring information, such as but not limited to host identity, current (and/or past) drug concentration, the location, correlation of the drug concentration with preprogrammed parameters, alerts, instructions/recommendations, and the like. In some embodiments, the communication device is configured to receive and process the drug information/location and to provide an alert/message to the user if/when the host's drug concentration meets a parameter. For example, the user may want to program the communication device to provide an alert only when the host consumes the drug that the ambulatory host monitor has been configured to detect. In some embodiments, the system can be configured to provide an alert/instructions/information if/when the host connects his ambulatory host monitor to a secondary device (e.g., a car). For example, in an embodiment wherein the ambulatory host monitor is configured to monitor alcohol consumption, the system is configured to transmit a notice/alert/drug information, etc., when the host attempts to start his car and has plugged his ambulatory host monitor into his car's ignition control device. In some circumstances, a single communication device can be configured to receive drug information/locations from a plurality of ambulatory host monitors (e.g., each applied to a different host), such that a user can monitor two or more hosts concurrently.

### Continuous In Vivo Hormone Monitoring

Another aspect provides a system configured for monitoring a hormone level in a host. Hormone level determination is conducted in a number of settings, such as but not limited to a clinical endocrinology setting, a fertility clinic setting, an obstetrics/gynecology setting, and in the home. For example, the relative levels of one or more of a woman's sex hormones can be monitored to determine if and/or when ovulation occurs (e.g., either to become pregnant or to avoid pregnancy), if the woman is pregnant, if menopause is complete, or if there is a hormonal imbalance that may be the cause or and/or secondary to an illness. In another example, secretion of hormones such as human growth hormone (hGH), insulin-like growth factor (IGF), thyroid hormones, insulin, factors that interact with hormones, and the like are measured in the clinic, such as to determine if the host has a hormonal abnormality.

Components of a Continuous In Vivo Hormone Monitoring System

In preferred embodiments, the system 10 includes a continuous analyte sensor 100 configured to detect a signal associated with a hormone concentration (or a signal associated with a factor associated with a hormone, such as but not limited to a binding protein, a reactant, a reaction product, a cofactor, etc.) in vivo. As described elsewhere herein, in preferred embodiments, the continuous analyte sensor is configured to detect a signal associated with a concentration of the hormone using any means, such as but not limited to electrochemistry, immunochemistry, radiochemistry, physical and/or chemical detection methods, optical detection methods, and combinations thereof. In various embodiments, the continuous analyte sensor is configured for invasive or noninvasive application to the host. For example, the continuous analyte sensor can be transcutaneous, wholly implantable, invertible into the host's circulatory system, or configured remain outside the host's body, such as to detect the analyte through the host's skin. In some embodiments, the continuous analyte sensor is configured to be wholly disposable. In other embodiments, the continuous analyte sensor is configured such that at least a part thereof is reusable (e.g., the transcutaneous electrodes and connectors for connecting the electrodes to electronics are disposable but the electronics are reusable).

In preferred embodiments, the system includes a communication device 110, as described elsewhere herein. The communication device includes electronics as described with reference to FIG. 2. In particular, the communication device includes a processor module configured to process the signal to provide hormone information. Hormone information includes but is not limited to the hormone's identity, the current concentration, changes in hormone concentration, trend and rate of change information, and information related to an event, such as but not limited to a predicted time of ovulation. In some embodiments, hormone information can include times of hormone secretion and clearance. In some embodiments, the system is configured to monitor two or more hormones. In these embodiments, hormone information can include information related to the concentrations of the two or more hormones and how changes/fluctuations therein are related. In preferred embodiments, the communication device is configured to output the hormone information, such as via a user interface. Preferably, the communication device is configured to output the hormone information in real time. Depending upon the system configuration, portions of the communication device can be located variously on the continuous analytes sensor, as a separate device carried by the host, or remotely, such as in a doctor's office or clinic. Depending upon the configuration, the continuous analyte sensor is operably connected to the communication device by either wired or wireless means. For example, the system can be configured such that the host wears the continuous analyte sensor, which includes electronics sufficient to power the sensor on her body and carries the remaining portion of the communications device (e.g., in a housing) in her pocket, wherein the sensor and the communications device are operably connected by radio frequency communication. In another example, the system can be configured such that the sensor is applied to the host in a clinical setting, and the sensor is wired to (e.g., plugged in to) the communication device adjacent to the host's chair/bedside/treadmill, etc.

Hormone secretion varies widely, depending upon the host's sex and age, including between hosts of a given cohort. Some hormones are continuously secreted at a rate that can vary over days, weeks, months or even years. Some hormones are released sporadically, as a surge, in response to circadian rhythms or stimulation. Other hormones are secreted at a basal level during certain periods and secretion surges at other periods. In some circumstances, it is desirable to store hormone information over time for a variety of purposes, such as but not limited to for evaluation of hormonal fluctuations over time and/or retrospective analysis. Accordingly, in some embodiments, the communication device is configured to store the hormone information over a period of time, such as but not limited to a period of hours, days, weeks, months or even longer. In preferred embodiments, the processor module is configured to process the stored hormone information together with the real-time hormone information (e.g., recently received hormone information) to provide diagnostic information.

In one exemplary embodiment, the system is configured to predict when a woman is ovulating. In general, ovulation occurs during a small window of time approximately in the middle of a woman's menstrual cycle. This window of time (which varies among women) can be accurately estimated by monitoring the woman's luteinizing hormone (LH) levels, which is relatively low during most of her cycle and surges a few days prior to ovulation. Accordingly, in preferred embodiments, the hormone detected is LH and the diagnostic information includes a time period associated with ovulation in the host. In some embodiments, the diagnostic information includes an alert, recommendation and/or instruction. For example, the system can be configured to provide an auditory, visual or tactile alert that ovulation is predicted to occur during an approaching window of time. An alert, recommendation and/or instruction can include information and/or instructions preprogrammed by a physician or by the manufacturer. For example, if the woman is using the system in a fertility clinic setting, the woman's physician might program the system to tell the woman to call the doctor when ovulation is about to occur or is occurring. Alternatively, in some circumstances, a system configured to monitor LH and provide information related to when ovulation occurs, which can be used by the host to avoid/prevent occurrence of pregnancy.

In another exemplary embodiment, the system is configured to determine if and/or when a woman becomes pregnant. In this embodiment, the hormone is human chorionic gonadotropin (HCG), which is secreted only during pregnancy, and the diagnostic information comprises pregnancy information. In some embodiments, the system is configured to monitor the host for the occurrence of both ovulation and pregnancy. For example, a woman using a system configured to monitor both LH and HCG, such as in a fertility clinic setting, can use the device to monitor when she ovulates and subsequently if she has become pregnant. In some circumstances, a system configured to monitor LH and/or HCG can provide diagnostic information that can be used (e.g., by a physician) to determine if a woman has a hormonal dysfunction, such as if the woman has difficulty becoming pregnant and/or maintaining a pregnancy.

In other embodiments, the system can be configured to detect a variety of hormones, such as but not limited to estradiol, progesterone, follicle stimulating hormone, follicle stimulating hormone β subunit, thyroid stimulating hormone, testosterone, human chorionic gonadotropin, and insulin.

Method of Ovulation Detection

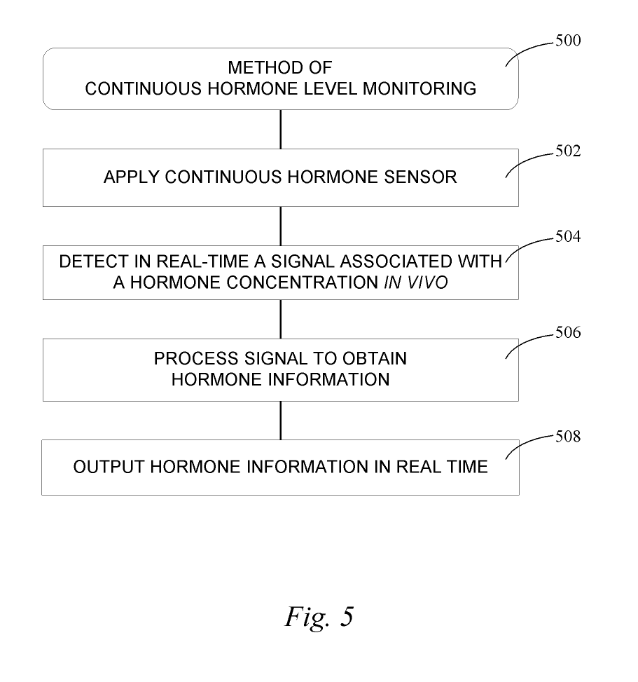

FIG. 5 is a flow chart 500 illustrating a method of monitoring a hormone level continuously.

At block 502, a continuous hormone sensor, configured to detect a signal associated with a concentration of a hormone in vivo is applied to the host 8 (e.g., implanted in the host, such as but not limited transcutaneously). The hormone can be any hormone of interest, such as but not limited to luteinizing hormone, human chorionic gonadotropin, estradiol, progesterone, follicle stimulating hormone, follicle stimulating hormone β subunit, thyroid stimulating hormone, testosterone, human chorionic gonadotropin, and insulin. In some embodiments, the sensor is configured to detect a signal associated with a cofactor, metabolite, or the like (associated with the hormone of interest) and which is indicative of the hormone's secretion. In some embodiments, the sensor is configured to detect the functionality of the hormone, such as to determine if the host's hormone secreted is functioning as a normal hormone would function. For example, the sensor can be configured to detect a signal associated with the binding of estrogen to the estrogen receptor; if the host's estrogen if functioning normally, a signal is detected; if the host's estrogen is not functioning normally, the signal will be reduced and/or absent entirely.

At block 504, a signal associated with the hormone concentration in vivo is detected in real-time. In other words, the signals are continuously and/or continually detected, such that the current hormone concentration can be determined at/during substantially any given time and/or period of time.

At block 506, the signal is processed to obtain hormone information. Since the system is configured to detect the signal in real-time, the processor can be configured to update the hormone information as quickly as the signal is received. As a result, the system can be configured to use the data to create continuously updated output.

At block 508, the system is configured to output the hormone information in real-time. Accordingly, the current hormone level can be displayed continuously on the user interface (e.g., the hormone information displayed is continuously updated). In some embodiments, the analyte sensor includes a display, such as a small LCD screen, and can display the current hormone level and/or a graphic indicative of a hormone concentration and/or an event (e.g., ovulation). In some embodiments, the information is displayed on a user interface associated with the communication device.

In some embodiments, the system is configured to store hormone information over a period of time. For example, the hormone information can be stored for a period of hours, days, weeks, or even months. In some embodiments, the system is configured to process the stored hormone information with real-time information, to provide diagnostic information. As a non-limiting example, in one embodiment, the system is configured to provide information related to a window of time during which a woman is likely to ovulate, such as to increase the likelihood of becoming pregnant. Accordingly, in this embodiment, the system is configured to store hormone information over a period of two or more months; the system is configured to process the stored hormone information to determine when the LH surge of the woman's menstrual cycle tends to occur. This information is processed with real-time hormone information, to determine when the next LH surge is likely to occur and/or if it is presently occurring, and the most likely window of time for ovulation to occur. In some embodiments, the system is configured to display the stored hormone information, such as a graph and/or table. In some embodiment, an ovulation window can be displayed graphically (e.g., as a graph or using symbols), as a table, and/or as text.

As a non-limiting example, in some embodiments, the system is configured for use in the diagnosis of some forms of human growth hormone (hGH) deficiency, such as a form of hGH deficiency wherein the hGH is not secreted in sufficient amounts to promote a predetermined level of growth in the host 8 having short stature, such as determined by the host's endocrinologist. It can be difficult to measure and/or monitor hGH levels in an individual, because hGH secretion generally occurs as several surges throughout the day, with low basal secretion (usually less than 3 ng/nL) for most of the day and night. Currently, to test an individual's ability to secrete hGH, simulation of secretion is attempted by exercise, insulin induced hypoglycemia, and/or injection of arginine, L-dopa, or clonidine. Unfortunately, these tests are often unsuccessful and/or inconclusive. Accordingly, in some embodiments, the system is configured to provide information related to the host's hGH levels. In this embodiment, the analyte sensor is configured to continuously detect a signal associated with an hGH concentration in the host. The processor module processes the signal and provides hormone information related to the host's hGH level in real-time. In some embodiments, the system is configured to store the hormone information, such that the stored information can be processed to provide diagnostic information. Preferably, the system is configured to output the stored and real-time hormone information and/or the diagnostic information, such as for use in diagnosis of the host's condition. For example, the system can be applied to a child suspected to have hGH deficiency. The child can wear the system continuously over a period of time, such as 1-10 days, during which the system continuously detects signals associated with the hGH levels in the child (and stores the information for later analysis by the child's endocrinologist/physician). Thus, hormone information related to the child's basal hGH secretion and surges can be gathered over time, without trying to stimulate an hGH surge. Such information provides the endocrinologist a more complete picture of the child's hGH metabolism, which enables a more accurate diagnosis of why the child is short. In some embodiments, the system is configured to detect other hormones in a similar manner, for diagnostic purposes.

Continuous In Vivo Nutrition Status Monitoring

Another aspect is a system configured for the continuous monitoring a host's nutrition status in vivo. A host's nutrition status can be monitored if the host is in long-term care, is elderly, has or is at risk of contracting a wasting disease, has cancer, has extensive severe burns, has undergone significant surgery, has a significant infection, has a chronic wound (e.g., impaired wound healing) or an acute wound (e.g., due to surgery) that the physician is concerned may become a chronic wound. With respect to chronic wounds, a chronic wound is a wound that does not heal in an orderly set of stages (e.g., deranged/impaired wound healing) within about three months. Chronic wounds may take years to heal and some never do so. These wounds occur most often in diabetics and people over the age of 60. Factors that contribute to poor wound healing include but are not limited to poor circulation, neuropathy, difficulty moving, systemic illnesses, poor nutritional status (e.g., protein-energy deficiency), high blood sugar levels (e.g., above about 135 mg/dl, diabetic), age (e.g., over 60), and significant and/or repeated trauma (e.g., due to surgery and/or injury). These wounds can cause severe emotional and physical stress to the patient and create a significant financial burden on patients and the whole healthcare system. Nutritional status can be evaluated by measuring a host's protein-energy level (e.g., serum albumin) and/or glucose level.

Components of a Continuous In Vivo Nutrition Status Monitor

Accordingly, in preferred embodiments, a monitoring device 10, including a continuous analyte sensor 100 configured and arranged for monitoring the nutritional status of a host 8, is provided. In preferred embodiments, the sensor includes a first sensing portion, a second sensing portion and a processor module. The first sensing portion is configured to measure a signal associated a glucose concentration (e.g., a first signal) in the host. The second sensing portion is configured to measure a signal associated with an albumin concentration (e.g., a second signal) in the host. The processor module is configured to process the first and second signals, to obtain in vivo nutrition information. Nutrition information includes but is not limited to information related to the concentration of a component of blood, serum, plasma, or interstitial fluid. In some embodiments, nutrition information includes but it not limited to information related to the concentration of albumin, urea, nitrogen and/or glucose in the host, such as concentration (current, previous or future), change in concentration, rate of change, acceleration of the change, trend information, a peak analyte concentration, a lowest analyte concentration, a correlation between a glucose concentration and an albumin concentration, and/or nutrition status. In some embodiments, nutrition information includes alerts, alarms, recommendations and/or instructions. In preferred embodiments, the system includes an output module configured to output the nutrition information (prospectively and/or retrospectively).

In preferred embodiments, the sensor is configured to use one or more detection mechanisms known in the art, including but not limited to electrochemical detection, immunochemical detection, physical detection, optical detection, radiological detection, or chemical detection. Accordingly, in some embodiments of the sensor, the albumin-sensing portion is configured and arranged to detect and/or measure a signal associated with the concentration of albumin using at least one of electrochemical detection, immunochemical detection, physical detection, optical detection, radiological detection, or chemical detection. In some embodiments, the albumin-sensing portion is configured to use a combination of these detection methods. Similarly, in some embodiments of the sensor, the glucose-sensing portion is configured and arranged to detect and/or measure a signal associated with the concentration of glucose using at least one of electrochemical detection, immunochemical detection, physical detection, optical detection, radiological detection, or chemical detection, or a combination thereof. In some embodiments, the albumin-sensing and glucose-sensing portions both use the same type of detection method. For example, in one embodiment, the albumin-sensing portion is configured to detect albumin via electrochemistry, and the glucose-sensing portion is also configured to detect glucose via electrochemistry. In other embodiments, the albumin-sensing and glucose-sensing portions use different detection method. For example, in one embodiment, the albumin-sensing portion is configured to detect albumin via immunochemistry, and the glucose-sensing portion is also configured to detect glucose via electrochemistry. Similarly, in some embodiments the albumin-sensing and glucose-sensing portions (or parts thereof) are both invasive or both non-invasive. In other embodiments, one sensing portion is invasive while the other sensing portion in non-invasive. If a sensing portion is invasive, it can be transcutaneous, intravascular or wholly implantable. In some embodiments, only a portion of a sensing portion is invasive. For example, in one embodiment, the sensing portion includes an electrode and an electronic component, wherein at least a portion of the electrode is configured for implantation in the host's body while the electronic component is configured to remain outside of the host's body.

In preferred embodiments, the continuous in vivo nutrition status monitor includes a communication device 110, as described elsewhere herein with reference to continuous medicament titration, continuous ambulatory drug monitoring, and/or continuous in vivo hormone monitoring. In some embodiments, the continuous in vivo nutrition status monitor is configured to operably connect to and/or integrate with a secondary device, as described elsewhere herein with reference to continuous medicament titration, continuous ambulatory drug monitoring, and/or continuous in vivo hormone monitoring.

Methods and devices that are suitable for use in conjunction with aspects of the preferred embodiments are disclosed in U.S. Pat. No. 4,994,167; U.S. Pat. No. 4,757,022; U.S. Pat. No. 6,001,067; U.S. Pat. No. 6,741,877; U.S. Pat. No. 6,702,857; U.S. Pat. No. 6,558,321; U.S. Pat. No. 6,931,327; U.S. Pat. No. 6,862,465; U.S. Pat. No. 7,074,307; U.S. Pat. No. 7,081,195; U.S. Pat. No. 7,108,778; U.S. Pat. No. 7,110,803; U.S. Pat. No. 7,192,450; U.S. Pat. No. 7,226,978; U.S. Pat. No. 7,310,544; U.S. Pat. No. 7,364,592; U.S. Pat. No. 7,366,556; U.S. Pat. No. 7,424,318; U.S. Pat. No. 7,471,972; U.S. Pat. No. 7,460,898; and U.S. Pat. No. 7,467,003.

Methods and devices that are suitable for use in conjunction with aspects of the preferred embodiments are disclosed in U.S. Patent Publication No. US-2005-0143635-1; U.S. Patent Publication No. US-2005-0181012-1; U.S. Patent Publication No. US-2005-0177036-1; U.S. Patent Publication No. US-2005-0124873-1; U.S. Patent Publication No. US-2005-0115832-1; U.S. Patent Publication No. US-2005-0245799-1; U.S. Patent Publication No. US-2005-0245795-1; U.S. Patent Publication No. US-2005-0242479-1; U.S. Patent Publication No. US-2005-0182451-1; U.S. Patent Publication No. US-2005-0056552-1; U.S. Patent Publication No. US-2005-0192557-1; U.S. Patent Publication No. US-2005-0154271-1; U.S. Patent Publication No. US-2004-0199059-1; U.S. Patent Publication No. US-2005-0054909-1; U.S. Patent Publication No. US-2005-0051427-1; U.S. Patent Publication No. US-2003-0032874-1; U.S. Patent Publication No. US-2005-0203360-1; U.S. Patent Publication No. US-2005-0090607-1; U.S. Patent Publication No. US-2005-0187720-1; U.S. Patent Publication No. US-2005-0161346-1; U.S. Patent Publication No. US-2006-0015020-1; U.S. Patent Publication No. US-2005-0043598-1; U.S. Patent Publication No. US-2005-0033132-1; U.S. Patent Publication No. US-2005-0031689-1; U.S. Patent Publication No. US-2004-0186362-1; U.S. Patent Publication No. US-2005-0027463-1; U.S. Patent Publication No. US-2005-0027181-1; U.S. Patent Publication No. US-2005-0027180-1; U.S. Patent Publication No. US-2006-0020187-1; U.S. Patent Publication No. US-2006-0036142-1; U.S. Patent Publication No. US-2006-0020192-1; U.S. Patent Publication No. US-2006-0036143-1; U.S. Patent Publication No. US-2006-0036140-1; U.S. Patent Publication No. US-2006-0019327-1; U.S. Patent Publication No. US-2006-0020186-1; U.S. Patent Publication No. US-2006-0036139-1; U.S. Patent Publication No. US-2006-0020191-1; U.S. Patent Publication No. US-2006-0020188-1; U.S. Patent Publication No. US-2006-0036141-1; U.S. Patent Publication No. US-2006-0020190-1; U.S. Patent Publication No. US-2006-0036145-1; U.S. Patent Publication No. US-2006-0036144-1; U.S. Patent Publication No. US-2006-0016700-1; U.S. Patent Publication No. US-2006-0142651-1; U.S. Patent Publication No. US-2006-0086624-1; U.S. Patent Publication No. US-2006-0068208-1; U.S. Patent Publication No. US-2006-0040402-1; U.S. Patent Publication No. US-2006-0036142-1; U.S. Patent Publication No. US-2006-0036141-1; U.S. Patent Publication No. US-2006-0036143-1; U.S. Patent Publication No. US-2006-0036140-1; U.S. Patent Publication No. US-2006-0036139-1; U.S. Patent Publication No. US-2006-0142651-1; U.S. Patent Publication No. US-2006-0036145-1; U.S. Patent Publication No. US-2006-0036144-1; U.S. Patent Publication No. US-2006-0200022-1; U.S. Patent Publication No. US-2006-0198864-1; U.S. Patent Publication No. US-2006-0200019-1; U.S. Patent Publication No. US-2006-0189856-1; U.S. Patent Publication No. US-2006-0200020-1; U.S. Patent Publication No. US-2006-0200970-1; U.S. Patent Publication No. US-2006-0183984-1; U.S. Patent Publication No. US-2006-0183985-1; U.S. Patent Publication No. US-2006-0195029-1; U.S. Patent Publication No. US-2006-0229512-1; U.S. Patent Publication No. US-2006-0222566-1; U.S. Patent Publication No. US-2007-0032706-1; U.S. Patent Publication No. US-2007-0016381-1; U.S. Patent Publication No. US-2007-0027370-1; U.S. Patent Publication No. US-2007-0032718-1; U.S. Patent Publication No. US-2007-0059196-1; U.S. Patent Publication No. US-2007-0066873-1; U.S. Patent Publication No. US-2007-0197890-1; U.S. Patent Publication No. US-2007-0173710-1; U.S. Patent Publication No. US-2007-0163880-1; U.S. Patent Publication No. US-2007-0203966-1; U.S. Patent Publication No. US-2007-0213611-1; U.S. Patent Publication No. US-2007-0232879-1; U.S. Patent Publication No. US-2007-0235331-1; U.S. Patent Publication No. US-2008-0021666-1; U.S. Patent Publication No. US-2008-0033254-1; U.S. Patent Publication No. US-2008-0045824-1; U.S. Patent Publication No. US-2008-0071156-1; U.S. Patent Publication No. US-2008-0086042-1; U.S. Patent Publication No. US-2008-0086044-1; U.S. Patent Publication No. US-2008-0086273-1; U.S. Patent Publication No. US-2008-0083617-1; U.S. Patent Publication No. US-2008-0119703-1; U.S. Patent Publication No. US-2008-0119704-1; U.S. Patent Publication No. US-2008-0119706-1. U.S. Patent Publication No. US-2008-0194936-1; U.S. Patent Publication No. US-2008-0194937-1; U.S. Patent Publication No. US-2008-0195967-1; U.S. Patent Publication No. US-2008-0183061-1; U.S. Patent Publication No. US-2008-0183399-1; U.S. Patent Publication No. US-2008-0189051-1; U.S. Patent Publication No. US-2008-0214918-1; U.S. Patent Publication No. US-2008-0194938-1; U.S. Patent Publication No. US-2008-0214915-1; U.S. Patent Publication No. US-2008-0194935-1; U.S. Patent Publication No. US-2008-0188731-1; U.S. Patent Publication No. US-2008-0242961-1; U.S. Patent Publication No. US-2008-0208025-1; U.S. Patent Publication No. US-2008-0197024-1; U.S. Patent Publication No. US-2008-0200788-1; U.S. Patent Publication No. US-2008-0200789-1; U.S. Patent Publication No. US-2008-0200791-1; U.S. Patent Publication No. US-2008-0228054-1; U.S. Patent Publication No. US-2008-0228051-1; U.S. Patent Publication No. US-2008-0262469-1; U.S. Patent Publication No. US-2008-0108942-1; U.S. Patent Publication No. US-2008-0306368-1; U.S. Patent Publication No. US-2009-0012379-1; U.S. Patent Publication No. US-2008-0287765-1; U.S. Patent Publication No. US-2008-0287764-1; U.S. Patent Publication No. US-2008-0287766-1; U.S. Patent Publication No. US-2008-0275313-1; U.S. Patent Publication No. US-2008-0296155-1; U.S. Patent Publication No. US-2008-0306434-1; U.S. Patent Publication No. US-2008-0306444-1; U.S. Patent Publication No. US-2008-0306435-1; and U.S. Patent Publication No. US-2009-0018424-1.

Methods and devices that are suitable for use in conjunction with aspects of the preferred embodiments are disclosed in U.S. patent application Ser. No. 09/447,227 filed Nov. 22, 1999 and entitled “DEVICE AND METHOD FOR DETERMINING ANALYTE LEVELS”; U.S. patent application Ser. No. 11/654,135 filed Jan. 17, 2007 and entitled “POROUS MEMBRANES FOR USE WITH IMPLANTABLE DEVICES”; U.S. patent application Ser. No. 11/654,140 filed Jan. 17, 2007 and entitled “MEMBRANES FOR AN ANALYTE SENSOR”; U.S. patent application Ser. No. 12/103,594 filed Apr. 15, 2008 and entitled “BIOINTERFACE WITH MACRO- AND MICRO-ARCHITECTURE”; U.S. patent application Ser. No. 12/055,098 filed Mar. 25, 2008 and entitled “ANALYTE SENSOR”; U.S. patent application Ser. No. 12/054,953 filed Mar. 25, 2008 and entitled “ANALYTE SENSOR”; U.S. patent application Ser. No. 12/133,789 filed Jun. 5, 2008 and entitled “INTEGRATED MEDICAMENT DELIVERY DEVICE FOR USE WITH CONTINUOUS ANALYTE SENSOR”; U.S. patent application Ser. No. 12/139,305 filed Jun. 13, 2008 and entitled “ELECTRODE SYSTEMS FOR ELECTROCHEMICAL SENSORS”; U.S. patent application Ser. No. 12/182,073 filed Jul. 29, 2008 and entitled “INTEGRATED RECEIVER FOR CONTINUOUS ANALYTE SENSOR”; U.S. patent application Ser. No. 12/247,137 filed Oct. 7, 2008 and entitled “IMPLANTABLE ANALYTE SENSOR”; U.S. patent application Ser. No. 12/250,918 filed Oct. 14, 2008 and entitled “ANALYTE SENSOR”; U.S. patent application Ser. No. 12/253,125 filed Oct. 16, 2008 and entitled “SIGNAL PROCESSING FOR CONTINUOUS ANALYTE SENSOR”; U.S. patent application Ser. No. 12/253,120 filed Oct. 16, 2008 and entitled “SIGNAL PROCESSING FOR CONTINUOUS ANALYTE SENSOR”; U.S. patent application Ser. No. 12/253,064 filed Oct. 16, 2008 and entitled “SIGNAL PROCESSING FOR CONTINUOUS ANALYTE SENSOR”; U.S. patent application Ser. No. 12/252,996 filed Oct. 16, 2008 and entitled “SIGNAL PROCESSING FOR CONTINUOUS ANALYTE SENSOR”; U.S. patent application Ser. No. 12/252,967 filed Oct. 16, 2008 and entitled “SIGNAL PROCESSING FOR CONTINUOUS ANALYTE SENSOR”; U.S. patent application Ser. No. 12/252,952 filed Oct. 16, 2008 and entitled “SIGNAL PROCESSING FOR CONTINUOUS ANALYTE SENSOR”; U.S. patent application Ser. No. 12/260,017 filed Oct. 28, 2008 and entitled “SENSOR HEAD FOR USE WITH IMPLANTABLE DEVICES”; U.S. patent application Ser. No. 12/258,320 filed Oct. 24, 2008 and entitled “SYSTEMS AND METHODS FOR PROCESSING SENSOR DATA”; U.S. patent application Ser. No. 12/263,993 filed Nov. 3, 2008 and entitled “SIGNAL PROCESSING FOR CONTINUOUS ANALYTE SENSOR”; U.S. patent application Ser. No. 12/264,835 filed Nov. 4, 2008 and entitled “IMPLANTABLE ANALYTE SENSOR”; U.S. patent application Ser. No. 12/258,235 filed Oct. 24, 2008 and entitled “SYSTEMS AND METHODS FOR PROCESSING SENSOR DATA”; U.S. patent application Ser. No. 12/258,345 filed Oct. 24, 2008 and entitled “SYSTEMS AND METHODS FOR PROCESSING SENSOR DATA”; U.S. patent application Ser. No. 12/258,325 filed Oct. 24, 2008 and entitled “SYSTEMS AND METHODS FOR PROCESSING SENSOR DATA”; U.S. patent application Ser. No. 12/258,318 filed Oct. 24, 2008 and entitled “SYSTEMS AND METHODS FOR PROCESSING SENSOR DATA”; U.S. patent application Ser. No. 12/258,335 filed Oct. 24, 2008 and entitled “SYSTEMS AND METHODS FOR PROCESSING SENSOR DATA”; U.S. patent application Ser. No. 12/264,160 filed Nov. 3, 2008 and entitled “DUAL ELECTRODE SYSTEM FOR A CONTINUOUS ANALYTE SENSOR”; U.S. patent application Ser. No. 12/267,542 filed Nov. 7, 2008 and entitled “ANALYTE SENSOR”; U.S. patent application Ser. No. 12/353,787 filed Jan. 14, 2009 and entitled “SYSTEMS AND METHODS FOR REPLACING SIGNAL ARTIFACTS IN A GLUCOSE SENSOR DATA STREAM”; U.S. patent application Ser. No. 12/353,799 filed Jan. 14, 2009 and entitled “SYSTEMS AND METHODS FOR REPLACING SIGNAL ARTIFACTS IN A GLUCOSE SENSOR DATA STREAM”; U.S. patent application Ser. No. 12/263,993 filed Nov. 3, 2008 and entitled “SIGNAL PROCESSING FOR CONTINUOUS ANALYTE SENSOR”; U.S. patent application Ser. No. 12/335,403 filed Dec. 15, 2008 and entitled “DUAL ELECTRODE SYSTEM FOR A CONTINUOUS ANALYTE SENSOR”; U.S. patent application Ser. No. 12/267,518 filed Nov. 7, 2008 and entitled “ANALYTE SENSOR”; U.S. patent application Ser. No. 12/264,835 filed Nov. 4, 2008 and entitled “IMPLANTABLE ANALYTE SENSOR”; U.S. patent application Ser. No. 12/273,359 filed Nov. 18, 2008 and entitled “TRANSCUTANEOUS ANALYTE SENSOR”; U.S. patent application Ser. No. 12/329,496 filed Dec. 5, 2008 and entitled “TRANSCUTANEOUS ANALYTE SENSOR”; U.S. patent application Ser. No. 12/359,207 filed Jan. 23, 2008 and entitled “TRANSCUTANEOUS ANALYTE SENSOR”; U.S. patent application Ser. No. 12/353,870 filed Jan. 14, 2009 and entitled “TRANSCUTANEOUS ANALYTE SENSOR”; U.S. patent application Ser. No. 12/267,525 filed Nov. 7, 2008 and entitled “ANALYTE SENSOR”; U.S. patent application Ser. No. 12/267,548 filed Nov. 7, 2008 and entitled “ANALYTE SENSOR”; U.S. patent application Ser. No. 12/267,547 filed Nov. 7, 2008 and entitled “ANALYTE SENSOR”; U.S. patent application Ser. No. 12/267,546 filed Nov. 7, 2008 and entitled “ANALYTE SENSOR”; U.S. patent application Ser. No. 12/267,544 filed Nov. 7, 2008 and entitled “ANALYTE SENSOR”; U.S. patent application Ser. No. 12/267,545 filed Nov. 7, 2008 and entitled “ANALYTE SENSOR”; U.S. patent application Ser. No. 12/267,494 filed Nov. 7, 2008 and entitled “INTEGRATED DEVICE FOR CONTINUOUS IN VIVO ANALYTE DETECTION AND SIMULTANEOUS CONTROL OF AN INFUSION DEVICE”; and U.S. patent application Ser. No. 12/267,531 filed Nov. 7, 2008 and entitled “ANALYTE SENSOR.”

All references cited herein, including but not limited to published and unpublished applications, patents, and literature references, are incorporated herein by reference in their entirety and are hereby made a part of this specification. To the extent publications and patents or patent applications incorporated by reference contradict the disclosure contained in the specification, the specification is intended to supersede and/or take precedence over any such contradictory material.

The term “comprising” as used herein is synonymous with “including,” “containing,” or “characterized by,” and is inclusive or open-ended and does not exclude additional, unrecited elements or method steps.

All numbers expressing quantities of ingredients, reaction conditions, and so forth used in the specification are to be understood as being modified in all instances by the term “about.” Accordingly, unless indicated to the contrary, the numerical parameters set forth herein are approximations that may vary depending upon the desired properties sought to be obtained. At the very least, and not as an attempt to limit the application of the doctrine of equivalents to the scope of any claims in any application claiming priority to the present application, each numerical parameter should be construed in light of the number of significant digits and ordinary rounding approaches.

The above description discloses several methods and materials of the present invention. This invention is susceptible to modifications in the methods and materials, as well as alterations in the fabrication methods and equipment. Such modifications will become apparent to those skilled in the art from a consideration of this disclosure or practice of the invention disclosed herein. Consequently, it is not intended that this invention be limited to the specific embodiments disclosed herein, but that it cover all modifications and alternatives coming within the true scope and spirit of the invention.

What is claimed is:

1. A system for providing information associated with a titration of a medicament in a host, comprising:
a continuous analyte sensor configured to detect a first signal associated with a medicament concentration in vivo in a host; and
a communication device comprising an input module configured to receive titration parameters, and a processor module configured to process the first signal and the titration parameters to obtain titration information associated with a titration of the medicament, wherein the communication device is configured to output the titration information.

2. The system of claim 1, wherein the titration parameters comprise at least one parameter selected from the group consisting of medicament identity information, a target medicament concentration, a medicament concentration limit, a toxic medicament concentration, a medicament delivery rate, a medicament delivery time, host data, and medicament effect information.

3. The system of claim 2, wherein the processor module is configured to provide an alarm when the medicament concentration is substantially within a predetermined percentage of the medicament concentration limit.

4. The system of claim 1, wherein the titration information comprises at least one member selected from the group consisting of a current medicament concentration, a predicted medicament concentration, a change in medicament concentration, an acceleration of medicament concentration, a relationship of medicament concentration and a medicament concentration limit, rate of change information, a clearance rate, and a correlation between a medicament concentration and a medicament effect experienced by the host.

5. The system of claim 1, wherein the information comprises at least one member selected from the group consisting of a therapy recommendation and a therapy instruction.

6. The system of claim 1, wherein the input module is further configured to receive a second signal associated with an effect of the medicament, and wherein the processor module is further configured to process the first signal, the second signal and the titration parameters to obtain the titration information.

7. The system of claim 6, further comprising a secondary medical device.

8. The system of claim 7, wherein the secondary medical device comprises at least one device selected from the group consisting of a secondary analyte sensor and a patient monitor, wherein the secondary medical device is configured to detect a second signal associated with an effect of a delivered medicament.

9. The system of claim 8, wherein the effect of the delivered medicament is associated with a change in a host physical attribute.

10. The system of claim 8, wherein the medicament comprises an anti-multiple sclerosis medicament, and wherein the effect of the delivered medicament comprises a change in at least one member selected from the group consisting of a multiple sclerosis symptom and a side effect of the anti-multiple sclerosis medicament.

11. The system of claim 8, wherein the medicament comprises an anti-epilepsy medicament, and wherein the effect of the delivered medicament comprises a change in at least one member selected from the group consisting of an epilepsy symptom and a side effect of the anti-epilepsy medicament.

12. The system of claim 1, wherein the communication device is configured to output the titration information to a secondary medical device.

13. The system of claim 12, wherein the secondary medical device comprises an anesthesia device.

14. The system of claim 12, wherein the secondary medical device comprises a medicament delivery device.

15. The system of claim 12, wherein the secondary medical device is configured to monitor an attribute of the host.

16. The system of claim 1, wherein the processor module is configured to determine an optimal dose of the medicament.

17. The system of claim 1, wherein the communication device comprises a user interface configured to perform at least one of outputting the titration information and receiving titration parameters.

18. A system for continuous ambulatory drug testing, comprising:
an ambulatory host monitor comprising a continuous sensor configured to detect a signal associated with a presence of a drug in vivo in a host, a location module configured to provide a location of the continuous sensor, and a first processor module configured to process the signal to obtain drug information; and
a transmitter configured to transmit the drug information.

19. The system of claim 18, further comprising a communication device located remotely from the ambulatory host monitor, wherein the communication device is configured to receive the drug information and the location, and to process the drug information and the location to obtain drug-monitoring information, and wherein the communication device is configured to output the drug-monitoring information.

20. The system of claim 19, wherein the drug-monitoring information comprises at least one of an instruction and a recommendation.

21. The system of claim 18, wherein the first processor module is configured to provide an alarm when the signal is below a programmed level.

22. The system of claim 18, wherein the drug is a drug of abuse and wherein drug information comprises information associated with a presence of the drug of abuse in the host.

23. The system of claim 18, wherein the drug is a medicament and the drug information comprises information associated with a presence of the medicament in the host.

24. The system of claim 23, wherein the medicament comprises an anti-tuberculosis medicament.

25. The system of claim 18, further comprising a secondary device configured to operably connect with the ambulatory host monitor, wherein the ambulatory host monitor is further configured to provide drug information to the secondary device, and wherein the secondary device is configured to perform at least one of providing an alert and deactivating a machine.

26. The system of claim 18, wherein the continuous sensor is a transcutaneous continuous sensor.

27. A system for continuously monitoring a hormone level, comprising:
a continuous hormone sensor configured to detect a signal associated with a hormone concentration in vivo in a host; and
a communication device comprising a processor module configured to process the signal to provide hormone information, wherein the communication device is configured to output the hormone information in real time.

28. The system of claim 27, wherein communication device is further configured to store the hormone information over time, and wherein the processor module is further configured to process the stored hormone information and the real-time hormone information to provide diagnostic information.

29. The system of claim 28, wherein the hormone is luteinizing hormone, and wherein the diagnostic information comprises a time period associated with ovulation in the host.

30. The system of claim 27, wherein the hormone is human chorionic gonadotropin, and wherein the diagnostic information comprises pregnancy information.

31. The system of claim 27, wherein the sensor is configured to measure a signal associated with at least one hormone selected from the group consisting of luteinizing hormone, estradiol, progesterone, follicle stimulating hormone, follicle stimulating hormone β subunit, thyroid stimulating hormone, testosterone, and human chorionic gonadotropin.

32. An analyte sensor for monitoring nutritional status in a host, comprising:
a first sensing portion configured to measure a first signal associated with a glucose concentration in a host;
a second sensing portion configured to measure a second signal associated with an albumin concentration in the host; and
a processor module configured to process the first signal and the second signal to obtain nutrition information in vivo.

33. The device of claim 32, wherein the first sensing portion is configured and arranged to measure the first signal using at least one detection method selected from the group consisting of electrochemical detection, immunochemical detection, physical detection, optical detection, radiological detection, chemical detection, and combinations thereof.

34. The device of claim 32, wherein the second sensing portion is configured and arranged to measure the second signal using at least one detection method selected from the group consisting of electrochemical detection, immunochemical detection, physical detection, optical detection, radiological detection, chemical detection, and combinations thereof.

35. The device of claim 32, further comprising an output module configured to output the nutrition information.

36. The device of claim 35, wherein the nutrition information comprises at least one member selected from the group consisting of an analyte concentration, a change in analyte concentration, a rate of change in analyte concentration, a peak analyte concentration, a lowest analyte concentration, a correlation between a glucose concentration and an albumin concentration, nutrition status, and an alarm.
